(19) United States
(12) Patent Application Publication
(10) Pub. No.: US 2005/0112559 A1
(43) Pub. Date: May 26, 2005

(54) Compositions and methods for diagnosing and preventing severe acute respiratory syndrome (SARS)

(75) Inventors: Tze Ming Danny Leung, Ma On Shan (HK); Chi Hang Frankie Tam, Shatin (HK); Chun Hung Ma, Siu Sai Wan (HK); Pak Leong Lim, Ma On Shan (HK); Kay Sheung Paul Chan, North Point (HK)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE CHINESE UNIVERSITY OF HONG KONG

(21) Appl. No.: 10/954,815

(22) Filed: Sep. 29, 2004

Related U.S. Application Data
(60) Provisional application No. 60/507,207, filed on Sep. 29, 2003.

(51) Int. Cl. C12Q 1/70

(52) U.S. Cl. 435/5

(57) ABSTRACT

The present invention relates to the fields of immunology and molecular biology and describes compositions and methods for using proteins, peptides and nucleic acids related to the SARS CoV nucleocapsid protein and the spike glycoprotein. In particular, the present invention provides immunostimulatory preparations, prophylactic pharmaceutical preparations, diagnostic assays and kits for identifying and preventing SARS infections.

### CROSS-REFERENCES TO RELATED APPLICATIONS

THIS IS A CONTINUATION—IN-PART APPLICATION CLAIMING THE BENEFIT OF THE PROVISIONAL APPLICATION SER. No. 60/507,207, FILED Sep. 29, 2003, THE DISCLOSURE OF WHICH IS INCORPORATED HEREIN BY REFERENCE.

### FIELD OF THE INVENTION

The present invention relates to the fields of immunology and molecular biology and describes compositions and methods for using proteins, peptides and nucleic acids related to the SARS CoV nucleocapsid protein and the spike glycoprotein.

### BACKGROUND OF THE INVENTION

Severe acute respiratory syndrome (SARS) is a new infectious disease in humans caused by a novel coronavirus called SARS-CoV [Poutanen S M, et al., 2003. N Engl J. Med. Published at www.nejm.org Mar. 31, 2003; Peiris J S M, et al., 2003. Lancet 361, 1319-1325; Ksiazek T G, et al., 2003. N Engl J. Med. Published at www.nejm.org Apr. 10, 2003; and Drosten C, et al., 2003. N Engl J. Med. Published at www.nejm.org Apr. 10, 2003]. This RNA virus is quite distinct from other coronaviruses known to humans or animals based on the structure of its 29,751 bp genome [Marra M A, et al., 2003. Science. Published at www.sciencexpress.org May 1, 2003; and Rota P A, et al., 2003. Science. Published at www.sciencexpress.org May 1, 2003.]. However, like all coronaviruses, it has genes for polymerase and structural proteins termed: spike (S), envelope (E), membrane (M) and nucleocapsid (N). In addition, it has genes for another 17 proteins, mostly non-structural, and some putative.

Generally, coronaviruses infect cells via the spike glycoprotein, which binds to specific cell receptors (such as aminopeptidase N) in the cell [Bonavia A, et al., 2003. J Virol 77, 2530-2538]. Following initial attachment, the viral envelope fuses with the plasma membrane of the cell and a cascade of intracellular events follows, including the interaction between the M and N proteins [Narayanan K, et al., 2000. J Virol 74, 8127-8134], eventually resulting in the production of progeny virions.

Currently the disease is diagnosed by clinical presentation and radiographic evidence of pneumonia. The incubation period for SARS is generally between 2 and 7 days. Typically, the patient develops high fever and respiratory problems that include cough and difficulty in breathing. There is therefore an urgent need for simple, accurate laboratory methods that unambiguously detecting the virus.

There are presently three types of laboratory test for SARS CoV:
(a) Growth and identification of the virus from the patient's specimen in cell culture.
(b) Detection of genetic material from the virus in the patient's specimen by polymerase chain reaction (PCR) methodology.
(c) Detection of antibodies to the virus in the patient's blood.

Cell culture and PCR methods may be more reliable for detecting the virus in samples from patients in the early stages of SARS (i.e., the first week) but have been shown to be less reliable as the disease progresses. In contrast, serological detection of the virus is more reliable in later stages of the disease (i.e., after first week). Thus, the culture or PCR method and the antibody method are complementary to each other, and detection of all cases of SARS may require a combination of methods.

There are currently two types of serological methods available to the clinician. The indirect immunofluorescence assay (IF) detects binding of antibodies, from infected individuals sera, to monkey cells (Vero) infected with the SARS virus and fixed to a microscope slide. Virus-infected cells are typically prepared by individual laboratories, although commercial preparations have recently become available (Euroimmun, Luebeck, Germany). Detection of binding requires manual examination of the microscope slide. The IF test is therefore labor-intensive as each specimen must be examined by eye and subjectively determined to be a positive or negative result, making the test impracticable for high-throughput screening of large numbers of samples.

An alternative assay is the enzyme-linked immunosorbent assay (ELISA). The ELISA assay utilizes antigenic viral antigens fixed to a solid surface. The patient's serum is incubated with the antigen and binding of antibodies in the serum to the antigens detected, for example, using a calorimetric assay. Although the ELISA format is amenable to high-throughput methods, there are currently no commercially-available ELISA kits available. Moreover, the viral antigens used in the assay are difficult to prepare from live virus as the yield low, there is unavoidable batch-to-batch variation between preparations, and cultures of virus pose a health risk.

Of equal if not greater importance in combating SARS CoV infection is the development of prophylactic vaccines that prevent or attenuate the infection. Identifying antigenic proteins that raise an immune response to viral particles and/or viral-infected cells an important step in developing any such vaccine. To date, no viable protein-based vaccine against SARS CoV has been developed.

### BRIEF SUMMARY OF THE INVENTION

To address deficiencies in the diagnosis and prevention of SARS CoV, the present invention provides embodiments based on peptides and proteins cumulatively termed nucleocapsid antigen, and nucleic acids encoding the same. To aid in the diagnosis of SARS, the present invention provides method embodiments for detecting exposure to SARS-CoV using a sample from a patient. The method involves (a) contacting a biological sample to a protein comprising an amino acid sequence having at least about 75%, more preferably at least about 80%, 85%, 90%, most preferably at least about 95%, 98%, 99% or 100% sequence homology to SEQ ID NO:2 or SEQ ID NO:6 or both, preferably the amino acid sequence is that of SEQ ID NO:2 or SEQ ID NO:6 or both, more preferably the amino acid sequence is also recombinant; and, (b) detecting in the biological sample an antibody binding to the contacted protein, wherein binding of the antibody to the protein indicates the patient has been exposed to SARS-CoV. Aspects of this embodiment include using a protein comprising the amino acid of SEQ ID NO:2 alone or a combination of SEQ ID NO:2 and SEQ ID NO:6 proteins, which may be used in different proportions to each other. Additional aspects of this embodiment include optionally having the protein immobilized on a solid support, which is preferably formed from a plastic or a glass. Alternatively, the solid support is selected from the group consisting of microsphere, microplate and membrane.

The biological sample is selected from the group consisting of whole blood, serum, plasma, cerebrospinal fluid, colostrum, lymphatic fluid, breast milk, saliva, urine, nasal wipes, tears, mucus ascites fluid, semem, fecal matter, sputum, fetal fluid, and the like.

In some embodiments the protein contacting the biological sample is a recombinant protein that may be produced in bacteria.

In another embodiment of the invention, the protein contacting the biological sample is a segment of the amino acid sequence of SEQ ID NO:2 or SEQ ID NO:6 or both.

In some embodiments of the diagnostic method noted above, detecting antibody binding to the contacted protein involves contacting the antibody bound to the protein with a labeled molecule that specifically recognizes the antibody bound to the protein; and then detecting the labeled molecule. The label used can be any suitable label known in the art, for example, radioactive isotopes, fluorophores, chromophores, phosphors and enzymes. In preferred aspects of the invention, the label is an enzyme and the detecting step further comprises contacting the label with a molecule that is catalytically converted by the enzyme into a detectable (e.g., colored) product.

In situations where the protein is a component in a molecular mixture, the method of the invention comprises additional steps. The additional steps include separating the protein from other components of the molecular mixture; and transferring the protein to a solid support. Preferred solid supports include polyvinyl diflouride, nylon, and cellulose and derivatives thereof. In some aspects of this alternative method, separating the protein comprises electrophoresis of the molecular mixture through a porous support such as agarose, cellulose, porous silica and polyacrylamide.

Other embodiments of the invention provide prophylactic medicaments such as protein or peptide-based vaccines. Accordingly, the present invention provides a vaccine comprising a protein comrising an amino acid sequence having at least about 75%, more preferably at least about 80%, 85%, 90%, most preferably at least about 95%, 98%, 99% or 100% sequence homology to SEQ ID NO:2 or SEQ ID NO:6 or both or a segment thereof; and, a pharmaceutically acceptable excipient. In some aspects the vaccine also comprises an adjuvant. In other aspects of the embodiment, the protein is a fusion protein, which may be produced in a eukaryotic system. Still other aspects provide vaccine preparations including an antibiotic or antiviral drug.

In another embodiment of this invention, the vaccine comprises a protein which is fused to a protein comprising an amino acid sequence having at least about 75% sequence homology to an amino acid sequence selected from the group consisting of SEQ ID NO:10, SEQ ID NO:14 and SEQ ID NO:18. Alternatively, the vaccine comprises a fusion protein comprising immunogenic peptide comprising an amino acid sequence having at least about 10 contiguous amino acids selected from the group consisting of SEQ ID NO:10, SEQ ID NO:14 and SEQ ID NO:18, wherein the immunogenic peptide produces an immune response when introduced to a mammal systemically.

In addition to prophylactic proteinaceous vaccines, the present invention also provides live vaccine embodiments comprising a cell including a nucleic acid comprising a coding sequence for a first protein having at least about 75%, more preferably at least about 80%, 85%, 90%, most preferably at least about 95%, 98%, 99% or 100% sequence homology to SEQ ID NO:2 or SEQ ID NO:6 or both, or a fragment thereof, where the coding sequence is operably linked to an expression system suitable for expressing the first protein in the cell. Some aspects of the live vaccines have a nucleic acid that further comprises a coding sequence for a second protein situated in-frame with the coding sequence of the peptide. Optionally, live vaccines may also include an adjuvant. In preferred aspects of the embodiment, the protein is secreted, where it may enter the extracellular space, or remain associated with the cell surface, preferably through interaction of a cell surface anchor with the cell surface.

To aid users of the invention in its practice, the present invention provides kits for detecting exposure to SARS-CoV. In a preferred embodiment of this invention, the kit, for example, includes a protein, which may be recombinant, comprising an amino acid sequence having at least about 75%, more preferably at least about 80%, 85%, 90%, most preferably at least about 95%, 98%, 99% or 100% sequence homology to SEQ ID NO:2 or SEQ ID NO:6 or both, preferably the amino acid sequence is SEQ ID NO:2 or SEQ ID NO:6 or both; and instructions for using the protein to detect anti-SARS antibodies in a biological sample. Some aspects of kit embodiments also include a solid support. Others optionally include one or more implements for collecting the sample, which may be from any body tissue, fluid or waste that may contain anti-nucleocapsid antigen antibody. Such samples include, but are not limited to, whole blood, serum, plasma, cerebrospinal fluid, colostrum, lymphatic fluid, breast milk, saliva, urine, nasal wipes, tears, mucus ascites fluid, semem, fecal matter, sputum, fetal fluid, and the like.

Other aspects of the embodiment are kits that also include a binding moiety specifically recognizing anti-SARS antibodies bound to the protein. Preferably the binding moiety is an antibody, more preferably a labeled antibody. Suitable labels for use with binding moieties include radioactive isotopes, fluorophores, chromophores, phosphors and enzymes. Particularly preferred labels are enzymes, and when enzyme labels are to be used with kit embodiments of the invention, the kit preferably includes a molecule that is catalytically converted by the enzyme into a detectable (e.g., colored) product.

Additional embodiments of the invention for diagnosing SARS include diagnostic devices for testing exposure to SARS CoV comprising a solid support having bound thereto a protein comprising an amino acid sequence having at least about 75% sequence homology to SEQ ID NO:2 or SEQ ID NO:6 or both, preferably the amino acid sequence is SEQ ID NO:2 or SEQ ID NO:6 or both. In some embodiments, the protein included is recombinant. The diagnostic device is suitable for detection of exposure to SARS-CoV in humans and in animals. Preferably these diagnostic devices have a solid support formed as a dipstick to ease their use. More preferably, the solid support is enclosed in a housing to protect the components from, for example, damage or contamination.

Diagnostic device embodiments of the invention may be part of kit embodiments. For example, some embodiments of the invention are kits comprising a device having a solid support bound thereto a protein comprising an amino acid sequence having at least about 75%, more preferably at least about 80%, 85%, 90%, most preferably at least about 95%, 98%, 99% or 100% sequence homology to SEQ ID NO:2 or SEQ ID NO:6 or both, and instructions for using the device. These kits may include a variety of optional components, as described for the kits noted above. A preferred option of the present kit embodiments is an antibody specifically recognizing the amino acid sequence.

Additional embodiments of the present invention include fusion proteins that find utility as both diagnostic and therapeutic reagents. Accordingly, the present invention provides a method of detecting exposure to SARS-CoV using a biological sample from a patient. The method includes contacting a biological sample to a fusion protein comprising amino acid sequences homologous to two SARS CoV proteins, the nucleocapsid protein and the spike glycoprotein. The nucleocapsid protein-derived portion has an amino acid sequence having at least about 75% more preferably at least about 80%, 85%, 90%, most preferably at least about 95%, 98%, 99% or 100% sequence homology to SEQ ID NO:2 or SEQ ID NO:6. This nucleocapsid protein-derived portion is covalently linked to a peptide derived from the spike glycoprotein amino acid sequence. The peptide comprises an amino acid sequence having at least about 10, more preferably at least about 12, 14 or 16, most preferably at least about 20 contiguous amino acids selected from the amino acid sequences SEQ ID NO:10, SEQ ID NO:14 and SEQ ID NO:18. Exposure to SARS CoV is determined by detecting an antibody in the biological sample binding to the contacted fusion protein.

The invention also includes as another embodiment, an immunostimulatory preparation. This preparation comprises the fusion protein described above, and a pharmaceutically acceptable excipient. In another aspect of this embodiment, the fusion protein comprises tetanus toxoid, diphtheria toxoid or CpG-oligonucleotides which may be chemically conjugated to an immunogenic peptide comprising an amino acid sequence having at least about 10 contiguous amino acids selected from the group consisting of SEQ ID NO:10, SEQ ID NO:14 and SEQ ID NO:18.

### BRIEF DESCRIPTION OF THE DRAWINGS

FIG. 1 depicts gels of viral antigens reactive with the serum of SARS patients. FIG. 1A depicts Coomassie-stained gels of viral antigens with a portion of one gel, CB, used for Western blot analysis. FIG. 1B is a Western blot of sera from several patients against a crude viral extract.

FIG. 2 compares the detection efficiency for various tests for detecting SARS CoV in SARS and non-SARS patient sera.

FIG. 3A is a schematic depicting the structure of the SARS-CoV virus showing the important antigens.

FIG. 3B is a map of recombinant antigens of the present study showing the size (number of amino-acids) in each antigen or antigen subunit.

FIG. 3C is an acrylamide gel of affinity-purified recombinant antigens showing the purity and abundance.

FIG. 4A is a graphic depicting the discrepancy between the rNa ELISA (recombinant N-terminal nucleocapsid) and the IF test, and the similarity between the rNa ELISA and the crude antigen ELISA.

FIG. 4B depicts some similarity between the rNb ELISA (recombinant C-terminal nucleocapsid) and the IF test, and the discrepancy between the rNb ELISA and the crude antigen ELISA or the rNa ELISA.

FIG. 4C illustrates the similarity between the ELISA using both the recombinant nucleocapsid antigen together (rNa+rNb) and the IF test.

FIG. 5A is a Western blot analysis of sera from 2 patients (S35 and S44) with a crude SARS CoV viral extract in the presence of various antigens used as an inhibitor.

FIG. 5B is a Western blot analysis of mouse sera against a crude viral extract. Sera 1, 2 and 3 were obtained individually from 3 BALB/c mice immunized (primary dose+1 booster) with rNa. U, serum from unimmunized mice (representative of 3 mice); S, serum from mice immunized (same protocol as with rNa, and using equivalent amounts of antigen) with rSa or rSb (representative of 3 mice in each group) of the antigen.

FIG. 5C is a graphical representation of ELISA results obtained by titrating the immune mouse sera used in FIG. 5B against the respective immunizing antigen (rNa-GST, rSc-GST or rSa-GST).

FIG. 6 is a graphical representation of the levels of antibodies reactive with the nucleocapsid, as determined in the rNa ELISA, in SARS patients who died from the disease and in those who survived. Also shown are the IF results.

FIG. 7 is a bar graph comparing the sensitivity of a commercially available ELISA test kit with an embodiment of the present invention.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“About” refers to a range of values of plus or minus 10% of the specified value. For example, the phrase “about 80%” includes plus or minus 10% of 80, or from 72 to 88.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, 7-carboxyglutamate, and o-phosphoserine. Amino acids may be referred to herein by either commonly known three letter symbols or by the one-letter symbols recommended by the IUPAC-IUB Biochemical Nomenclature Commission.

“Amino acid analog” refers to compounds that have the same basic chemical structure as a naturally occurring amino acid, i.e., a carbon that is bound to hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

“Amino acid sequence” refers to the positional relationship of amino acid residues as they exist in a given polypeptide or protein.

“Animal” includes, but is not limited to farm animals including cows, sheep, pigs, horses, goats and poultry (e.g., chickens, turkeys, ducks, fowl, game birds and geese) companion animals such as dogs and cats; exotic and/or zoo animals; and laboratory animals including mice, rats, rabbits, guinea pigs, and hamsters.

“Antibody” or “Functional antibody” refers to a polypeptide ligand substantially encoded by an immunoglobulin gene or immunoglobulin genes, or fragments thereof, which specifically binds and recognize an epitope (e.g., an antigen). Antibodies are structurally defined by the interaction of two forms of polypeptide, one termed an “antibody light chain” and the other termed an “antibody heavy chain”. Each antibody light chain is covalently bound to an antibody heavy chain through one or more covalent bonds termed disulfide bridges. Each disulfide bridge consists of a disulfide bond between the y-sulfide groups of two cysteine residues, one cysteine being part of the antibody heavy chain and the other cysteine being part of the antibody heavy chain. In addition to the covalent association with an antibody light chain, each antibody heavy chain can also be covalently associated with one or more antibody heavy chains. As with the association with antibody heavy and light chains, the interaction between two antibody heavy chains is through one or more disulphide bridges. The heavy chain defines the class of the antibody: IgM, IgG, IgA, IgD or IgE. IgM antibodies are found early in the serum in an immune response, and other classes, notably, IgG, later. IgG antibodies are generally produced in greater amounts than IgM antibodies in infections.

Generally, each antibody light chain and each antibody heavy chain is encoded in a separate transcriptional unit, or gene. The present invention however also envisions chimeric antibody genes encoding both heavy and light chains, including, but not limited to, chimeric genes where the coding sequences for heavy and light chains, two heavy chains, or a plurality of any combination of antibody heavy and light chains are joined by a nucleic acid encoding a linker peptide in-frame with the respective antibody-encoding sequences.

The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments discussed below.

The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

Antibodies can exist as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to a truncated heavy chain by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993)). While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Generally, a functional antibody is capable of specifically or selectively recognizing one or more epitopes found on an antigen. For example, an “antibody that specifically recognizes a product of the scorable homeostatic reporter element” is an antibody that under designated immunoassay conditions, binds to a protein encoded by a scorable homeostatic reporter element of the present invention with at least two times the background and does not substantially bind in a significant amount to other proteins that might be present in the sample. Typically a functional antibody will bind its antigen in a specific or selective reaction producing a signal at least twice that of the background signal or noise and more typically more than 10 to 100 times background, in a manner that is determinative of the presence of the antigen in a heterogeneous population of antigens and other biologics. anti-SARS antibodies.

An “anti-nucleocapsid antibody” is any antibody, as described herein, which specifically recognizes nucleocapsid antigen. Similarly, “anti-SARS CoV antibody” refers to any antibody that specifically recognizes an antigen associated with the SARS CoV virus.

“Antigenic” or “antigen” refers to substances which are capable, under appropriate conditions of inducing a specific immune response and of reacting with the products of that response, e.g., with specific antibodies or specifically sensitized T-lymphocytes, or both. Antigens may be soluble substances, such as nucleic acids, peptides or proteins, or particulates, such as bacteria and tissue cells; however, only the portion of the protein or polysaccharide molecule known as the antigenic determinant (epitopes) combines with antibody or a specific receptor on a lymphocyte.

“Antigenically neutral carrier protein” refers to proteins that are associated, covalently or noncovalently, with another molecule and do not stimulate an immune response when administered to a host organism.

An “antiviral drug” is any pharmaceutically acceptable composition that inhibits viral infectivity by at least 30%, more preferably 40%, 50%, 60%, 70%, 80%, or at least 90%, 95% or 98%.

A “biological sample” or “patient sample” refers to any sample taken from a living or dead organism. Examples of biological samples include, but are not limited to biological fluid specimen and biopsies. Biological fluid specimen include, but are not limited to whole blood, serum, plasma, cerebrospinal fluid, colostrum, lymphatic fluid, breast milk, saliva, urine, nasal wipes, tears, mucus ascites fluid, semem, fecal matter, sputum, fetal fluid and the like.

A “cell surface anchor” is any molecule capable of tethering itself and any associated molecular entity to the surface of a cell. The cell surface anchor may interact with any structure associated with the cell surface to accomplish this function, including covalent and non-covalent association.

The term “coding sequence,” in relation to nucleic acid sequences, refers to a plurality of contiguous sets of three nucleotides, termed codons, each codon corresponding to an amino acid as translated by biochemical factors according to the universal genetic code, the entire sequence coding for an expressed protein, or an antisense strand that inhibits expression of a protein. A coding sequence may be expressed if inserted into an appropriate expression system and introduced into a suitable host or in vitro expression system. A “genetic coding sequence” is a coding sequence where the contiguous codons are intermittently interrupted by non-coding intervening sequences, or “introns.” Thus, coding sequences include genomic sequences, both with and without introns, cDNA sequences, mRNA sequences and fragments thereof. During mRNA processing intron sequences are removed, restoring the contiguous codon sequence encoding the protein or anti-sense strand.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance for methods that depend upon binding between nucleic acids.

The term “expression system” refers to, at a minimum, all regulatory nucleotide sequences that necessarily must be operably linked to a coding sequence for the coding sequence to be expressed as protein. The term may also refer to optional regulatory nucleotide sequences that have the capacity to modulate protein expression from the coding sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to an amino acid sequence such as SEQ ID NO:2 or a nucleotide sequence such as SEQ ID NO:1 or SEQ ID NO:3), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of HIV envelope glycoproteins, fusion proteins comprising envelope glycoproteins and nucleic acid sequences encoding the same, the BLAST and BLAST 2.0 algorithms and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A molecule is said to be “immobilized,” for example to a surface, when the molecule is incapable of leaving the surface without a change in environmental conditions such as temperature, pressure, pH, ionic strength, or the molecule undergoes some form of chemical transformation, whether spontaneous or catalyzed. Typically molecules are immobilized to a solid support.

“Molecular mixture” refers to any composition of two or more molecularly distinct moieties whether in solid, gas or liquid phase.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-o-methyl ribonucleotides and peptide-nucleic acids (PNAs). Nucleotides may be referred to by their commonly accepted single-letter codes.

Unless otherwise indicated, a particular nucleic acid sequence also implicitly encompasses conservatively modified variants thereof (e.g., degenerate codon substitutions, see below) and complementary sequences, as well as the sequence explicitly indicated.

“Nucleocapsid antigen” refers to any peptide or protein comprising an amino acid sequence having at least about 75%, more preferably at least about 80%, 85%, 90%, most preferably at least about 95%, 98%, 99% or 100% amino acid sequence homology with SEQ ID NO:2 that, will raise an immune response at least 30%, more preferably at least 40%, 50%, 60%, 70%, 80%, 90%, or more as determined by quantitative ELISA testing or quantitative CTL assay as described in the art.

The phrase “operably linked” refers to a relational orientation of a promoter, terminator and/or control elements to a nucleic acid such that the nucleic acid is operably linked to a promoter, terminator and/or control elements allowing for transcription of the nucleic acid. The promoter, terminator and/or control elements of the construct constitute an “expression system.” Expression system may also be used in referring to promoter, terminator and/or control elements operably linked to a nucleic acid encoding a peptide or protein.

The terms “peptide” and “protein” are used herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer. Peptides and proteins of the present invention include amino acid polymers having D- and L-isoforms of individual amino acid residues, as well as other amino acid variants, as described herein. Peptides are distinguished by the number of amino acid residues making up the primary structure of the molecule. For purposes of this invention, typically, peptides are those molecules comprising up to 50 amino acid residues and proteins comprise 50 or more amino acid residues. However, methods of synthesis and/or delivery of peptides and proteins of the invention are similar, if not identical, as will be appreciated by one of skill in the art. Therefore, where appropriate, these terms are synonymous when discussing methods of synthesis, modification, expression or use as therapeutic or diagnostic reagents.

“Pharmaceutically acceptable excipient” refers to an inert substance used as a diluent or vehicle for a drug.

“Porous support” include, but is not limited to agarose, cellulose, porous silica and polyacrylamide.

“Sequence homology,” in the context of amino acid sequences, refers to the correspondence or resemblance of substances belonging to the same type or series; a similarity of composition varying by a small, regular difference, and usually attended by a regular variation in physical properties; as, there is a homology between glycine, alanine, leucine, etc. I.e., the term refers to two sequences differing in homologous amino acid changes in terms of the chemistry of the side groups of corresponding amino acids in the respective sequences.

As used herein, the term “solid support,” is used in its broadest sense to refer to a number of supports that are available and known to those of ordinary skill in the art. Solid supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid support may be selected on the basis of desired end use and suitability for various protocols. For example, solid supports for embodiments of the present invention include, for example for ELISA assays a plastic or a glass surface, or an inert bead; for western blotting, exemplary solid supports include polyvinyl diflouride, nylon, cellulose and derivatives thereof. Solid supports include a microsphere, a microplate, or a membrane. In some embodiments, the solid support may have a reactive surface or coating to aid in adhesion of molecular moieties. Solid supports of the present invention may have sealed or porous surfaces. In some embodiments, porous surfaces are preferred as they provide greater surface area for binding molecules of the invention. Exemplary porous supports suitable for use with the present invention include agarose and polyacrylamide gels, cellulose, and porous silica.

The phrase “stringent hybridization conditions” (or “stringent conditions”) refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

### INCORPORATION BY REFERENCE

All publications and patent applications cited in the specification are herein incorporated by reference as if each individual publication or patent application were specifically and individually indicated to be incorporated by reference.

### DETAILED DESCRIPTION OF THE INVENTION

I. Introduction

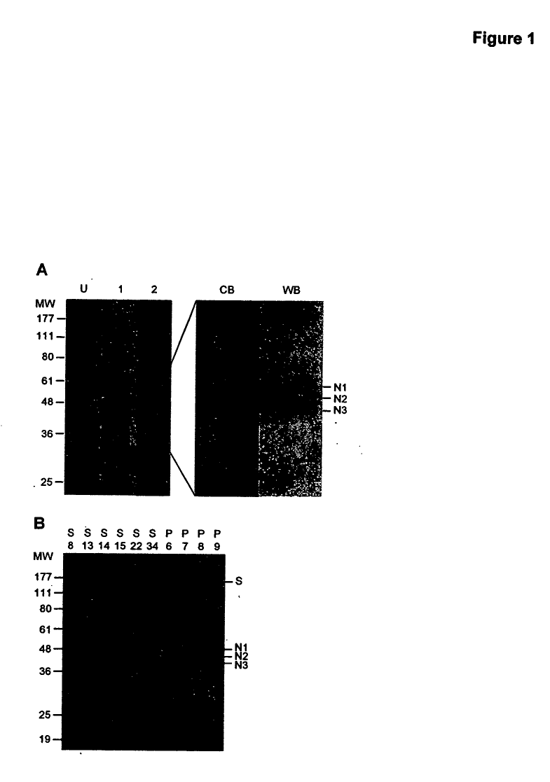

The present invention provides a SARS CoV-specific antigen, taken from the nucleocapsid protein of the virus, which has been found to be exceptionally reactive with antibody preparations taken from individuals who have been challenged with the SARS CoV virus. This is illustrated in FIG. 1, which identifies viral antigens that are reactive with the serum of SARS patients. FIG. 1(A) shows separation of crude mixtures of viral antigens obtained from virus-infected culture cells by electrophoresis and stained with a dye (Coomassie blue, CB). U, control uninfected cells. 1 and 2 are different preparation of infected cells; note the slight variation in protein (band) intensity at 36-48 kD between these preparations. WB, results of preparation #2 reacted with a SARS serum showing the highly reactive antigens, N1, N2 and N3. FIG. 1(B) provides additional Western blot results of 6 SARS and 4 non-SARS pneumonia patients, showing strong reactivities of the N1-N3 antigens, and lesser reactivities of the spike (S) protein, and the 80 kD and 60 kD proteins.

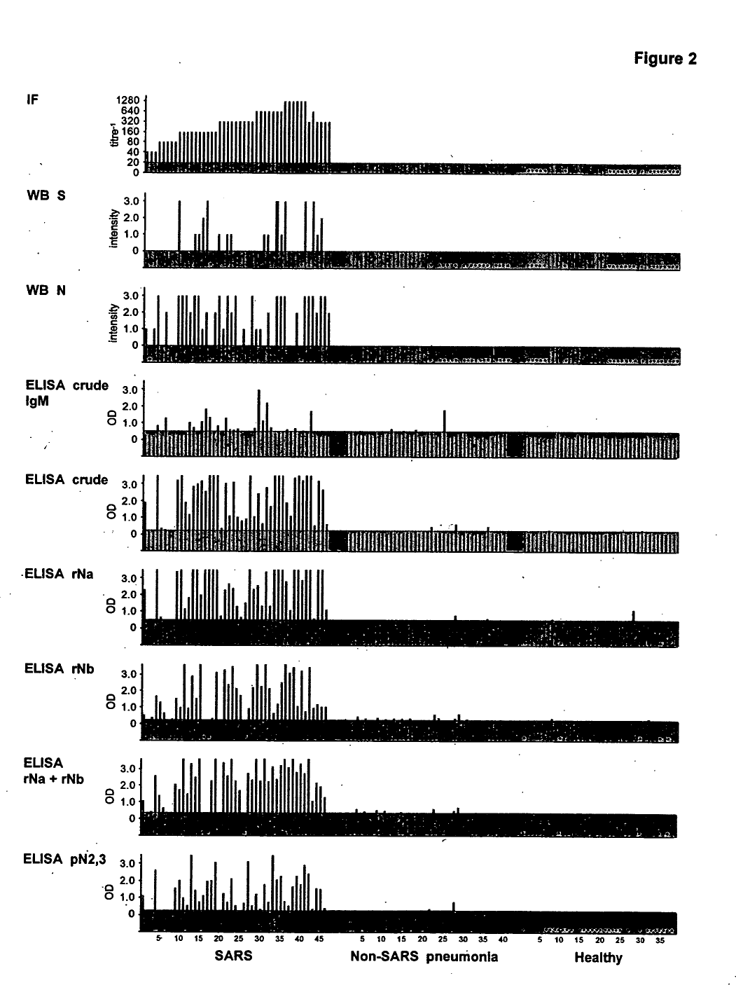

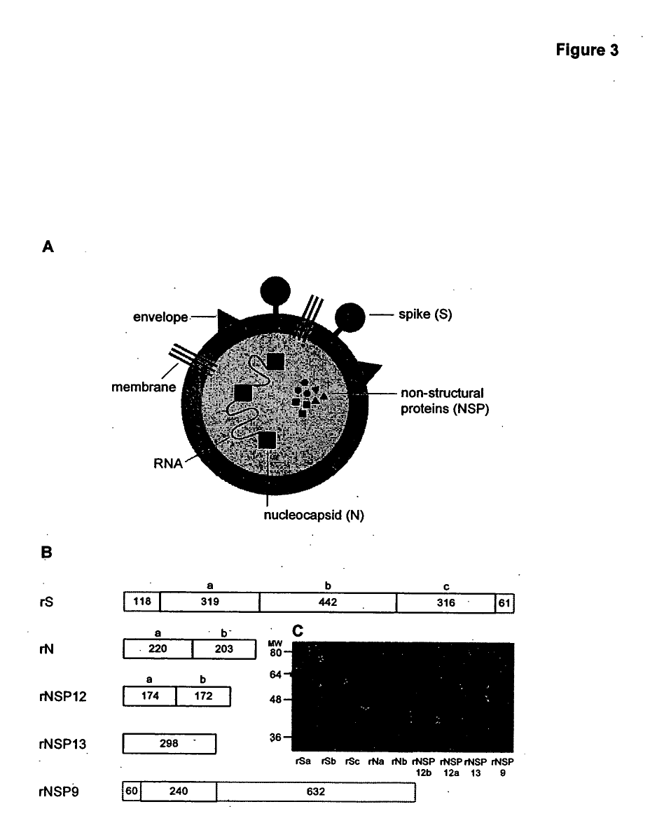

The diagnostic potential of the present invention is illustrated in FIG. 2, which compares the detection efficiency of various tests for SARS. Shown are the results of individual sera from each group of subjects (46 SARS patients, 40 non-SARS pneumonia patients and 38 healthy individuals) examined by the various tests based on IF, WB or ELISA. The antigens used are described in FIG. 3; I.e.: S, spike; N, nucleocapsid; crude, crude viral extract; rNa or rNb, recombinant nucleocapsid (subunit a or subunit b); p N2,3, the N2 and N3 antigens purified from the crude antigens by gel separation. Except in tests marked “IgM”, in which case only IgM antibodies were determined, all other tests were based on IgG antibody detection. In each test, positive results are those above the shaded bar. For ELISA assays, the cut-off for positive results is based on the mean value of the combined cohorts of non-SARS pneumonia and healthy subjects, plus 1 SD. In Western blots, the intensity of the reaction is arbitrarily scored by eye (3 is most intense). Sera that were not performed in a test are shown by a dot; the reason for their exclusion is the lack of antigen or serum, or the results were not readable.

FIG. 3A is a cartoon of the structure of the SARS-CoV virus showing the important antigens. FIG. 3B is a map of the bacterial recombinant antigens discussed herein, showing the size (number of amino-acids) in each antigen or its subunits. FIG. 3C is a gel analysis of the affinity purified recombinant antigens showing the purity and abundance.

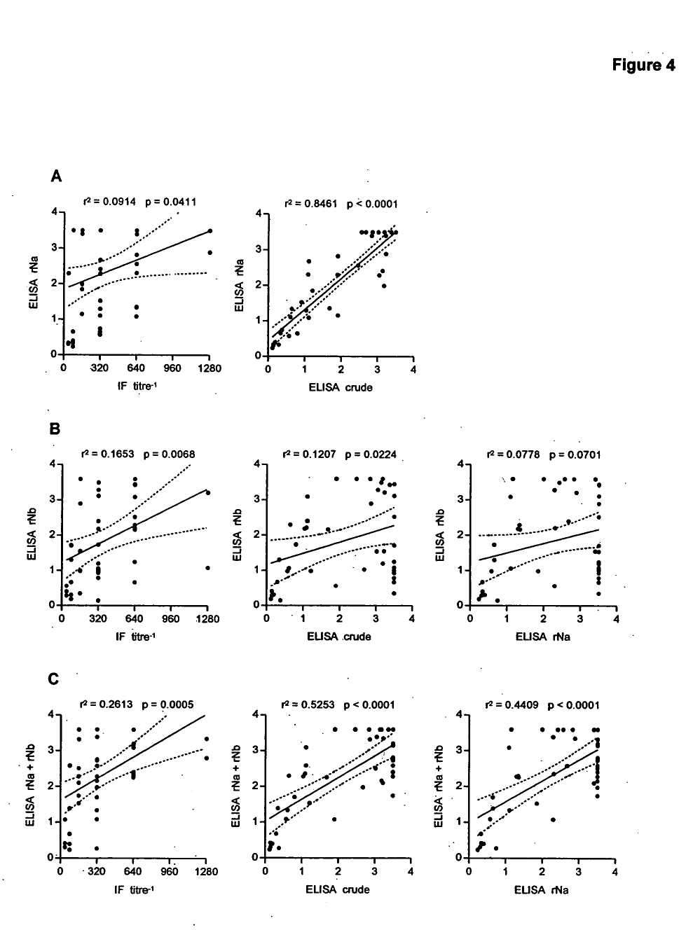

FIG. 4A shows the similarity between the N-terminal recombinant nucleocapsid antigen, rNa (SEQ ID NO:2), and the crude antigen from a cell lysate when compared using the ELISA method of the present invention, whereas much less similarity is shown between the N-terminal recombinant antigen ELISA and the IF test. FIG. 4B shows the reverse is the case with the C-terminal recombinant nucleocapsid antigen, rNb (SEQ ID NO:6). This suggests that antibodies are not made equally to the two nucleocapsid components (rNa and rNb) by patients (FIG. 4B, last diagram) and that the two nucleocapsid components are complementary to each other (FIG. 4C, first diagram, and FIG. 2). Indeed, the two components can be combined in different proportions so that an optimal ratio can be found to give the best results in terms of assay sensitivity and specificity. The rNb component is less specific than the rNa component and consequently a combination using much less of the rNb component was found to be ideal (data not shown).

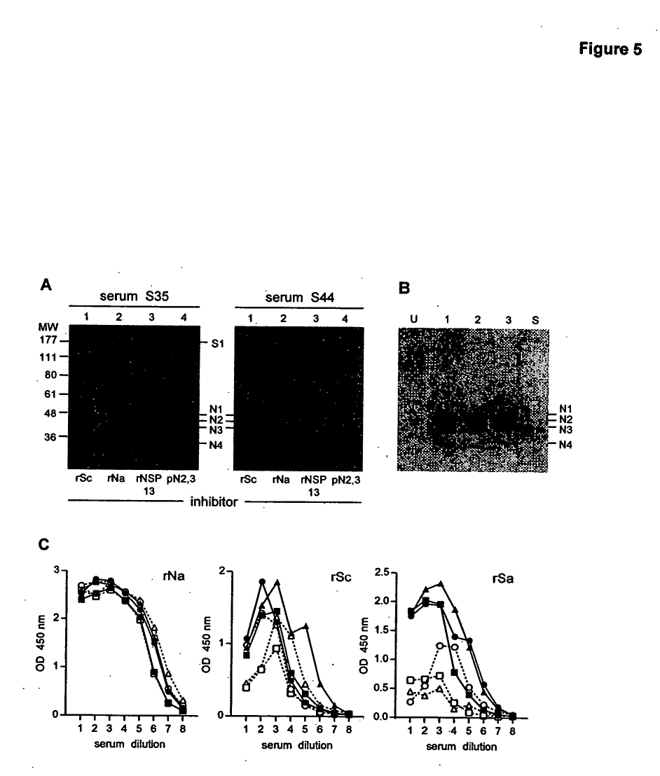

FIG. 5A provides evidence that the major reactive antigens of the virus, which are labeled N1, N2 and N3 in the Western blot assay, are all nucleocapsid antigens. Another nucleocapsid component, labeled N4, which is less reactive, is also found in the inhibition experiment shown. FIG. 5B confirms the results that N1, N2, N3 and N4 are nucleocapsid antigens using the sera of mice immunized previously with the N-terminal recombinant nucleocapsid antigen in Western blot analysis. The results can be interpreted to mean that N2, N3 and N4 are all derived from N1 through fragmentation by some cellular process, and increasing lengths of the C-terminal end of N1 are deleted from N2 to N4. This explains why the crude viral extract is more similar to the N-terminal recombinant nucleocapsid antigen than to the C-terminal recombinant nucleocapsid antigen.

FIG. 5C shows that the N-terminal recombinant nucleocapsid antigen, rNa (SEQ ID NO:2), is highly immunogenic in BALB/c mice, evidenced by the large amounts of antibodies found in the serum of these animals which are specific to the antigen used as vaccine (3 animals shown). The vaccine used has a carrier protein (GST), but very little of the antibodies are made to the carrier protein, shown by the lack of inhibition in the assay when GST is added to the serum (dashed line). In contrast, the recombinant spike antigens, rSa (SEQ ID NO:10) and rSc (SEQ ID NO:18), are only poorly immunogenic in BALB/c mice, and the antibodies elicited are made mostly to the carrier protein (high inhibition by GST).

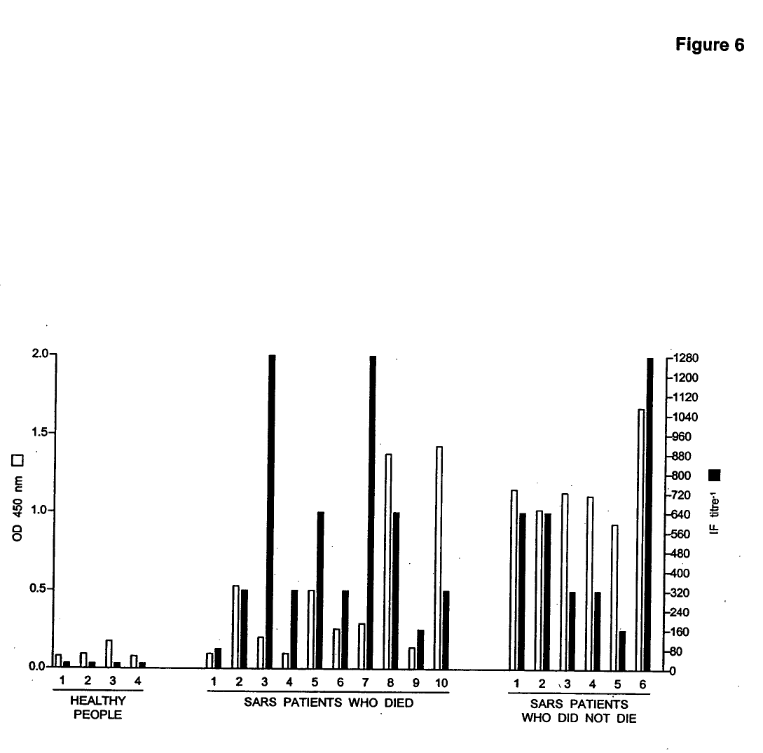

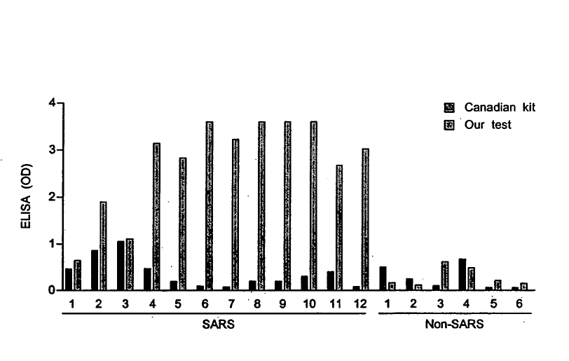

FIG. 6 shows eight of the ten patients who died from SARS had little antibodies to the N-terminal recombinant nucleocapsid antigen, whereas other SARS patients who survived had abundance of such antibodies. The dead patients, however, had other types of antibodies revealed in the IF test. This suggests that the nucleocapsid may be important in protection and useful as a vaccine.

From these results it is evident that the nucleocapsid antigen, has utility in diagnostic and prognostic methods or evaluating SARS infection and, due to its high antigenicity and immunogenicity, finds use as a prophylactic vaccine.

II. Preparing Nucleocapsid Antigens

As noted above, nucleocapsid antigens of the present invention find use as diagnostic reagents and as active ingredients in prophylactic medicaments. Antigen for either use may be isolated from any suitable source, and in some embodiments synthesized de novo using techniques well-known to those of skill in the art. For medicaments, viral-free preparations of antigen are desirable and recombinant methods are therefore preferred where the nucleocapsid antigen is expressed in isolation from other viral components. For diagnostic applications, recombinant methods also have health benefits over isolating the antigen from infectious viral particles found in biological samples (patient samples) or infected culture production.

A. Antigen Isolated from SARS-Infected Patients and Eukaryotic Cell Lines.

Fluid samples harvested from SARS-infected patients provide a ready, albeit hazardous, source of nucleocapsid antigen suitable for use in the present invention. Suitable biological samples containing SARS viral particles include, but are not limited to, whole blood, serum, plasma, cerebrospinal fluid, colostrum, lymphatic fluid, breast milk, saliva, urine, nasal wipes, tears, mucus ascites fluid, semem, fecal matter, sputum, fetal fluid and the like. Once the sample is obtained from the patient, the desired antigen may be isolated using techniques well-known it the art. Exemplary techniques suitable for isolating nucleocapsid antigen are described in detail below.

In some embodiments SARS-virus infected cell culture is used as a source of nucleocapsid antigen. Any suitable eukaryotic cell capable of supporting SARS virus production may be used, but Vero monkey cells are preferred. Any suitable culture procedure and culture medium also may be used to culture the cells in the process of the invention. Suitable culture procedures are well known and understood by those of skill in the art. Both serum-supplemented and serum-free media may be used. Batch and continuous fermentation procedures, suspension and adherent, e.g. microcarrier culture methods and stirred tank and airlift fermenters may be used as appropriate, having regard to cell type.

Infected cells may be grown to densities at or approaching maximum cell density in the case of suspension cultures, or to or approaching confluence in the case of adherent cell lines at which stage they may be transferred to a maintenance medium.

Protein production during the culture may be monitored by general assay techniques such as enzyme linked immunosorbent assay or immunoradiometric assay adapted for the particular protein in question. Where necessary, the protein may be purified by removal of extraneous material, particularly removal of cell and viral-derived molecules.

B. Recombinant Production of Nucleocapsid Antigen

Both eukaryotic and prokaryotic-based protein expression systems are contemplated by the invention, and their construction is well-known to those of skill in the art. However, preferred embodiments of the present invention take advantage of the surprising result that N-terminal fragments of the nucleocapsid protein expressed in bacteria are readily recognized by antibodies produced in response to SARS challenge. Moreover, nucleocapsid antigen expressed in bacteria is competent for producing an anti-SARS CoV immune response. The following sections provide additional teaching for creating the preferred bacterial-based expression systems of the invention.

1. Nucleic Acid Synthesis

Nucleic acids encoding the nucleocapsid antigen of the present invention may be constructed using any suitable method known to one of skill in the art. Basic texts disclosing methods for isolating native nucleocapsid-encoding nucleic acids using recombinant techniques include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Nucleic acids may be chemically synthesized according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Purification of nucleic acids is by either native acrylamide gel electrophoresis or by anion-exchange HPLC as described in Pearson & Reanier, J. Chrom., 255:137-149 (1983).

In addition to native nucleic acids encoding nucleocapsid antigen, the present invention also includes nucleocapsid antigen-encoding nucleic acids that vary from the native sequences, such as SEQ ID NO:1 or SEQ ID NO:5. For example the present invention contemplates nucleocapsid antigen-encoding nucleic acids that have at least about 75%, more preferably at least about 80%, 85%, 90%, most preferably at least about 95%, 98%, 99% or 100% nucleotide sequence identity with SEQ ID NO:1 or SEQ ID NO:5 or both. Where desirable, one of skill in the art will recognize many ways of generating alterations in a given nucleic acid sequence. Such well-known methods include site-specific mutagenesis, PCR amplification using degenerate nucleic acids, exposure of cells containing the nucleic acid to mutagenic agents or radiation, chemical synthesis of a desired nucleic acid (e.g., in conjunction with ligation and/or cloning to generate large nucleic acids) and other well-known techniques. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991).

Nucleic acids of the invention may include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, olate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference.

For example, covalent linkage of a cholesterol moiety to a nucleic acid can improve cellular uptake by 5- to 10-fold which in turn improves DNA binding by about 10-fold (Boutorin et al., 1989, FEBS Letters 254: 129-132). Ligands for cellular receptors may also have utility for improving cellular uptake, including, e.g. insulin, transferrin and others. Similarly, derivatization of oligonucleotides with poly-L-lysine can aid nucleic acid uptake by cells (Schell, 1974, Biochem. Biophys. Acta 340: 323, and Lemaitre, et al., 1987, Proc. Natl. Acad. Sci. USA 84: 648).

The sequence of nucleic acids of the present invention may be verified using, e.g., the chain termination method for sequencing double-stranded templates of Wallace et al., Gene, 16:21-26 (1981) or using the chemical degradation method of Maxam and Gilbert (1980) in Grossman and Moldave (eds.) Academic Press, New York, Methods in Enzymology 65:499-560. Sequences of short oligonucleotides can also be analyzed by laser desorption mass spectroscopy or by fast atom bombardment (McNeal, et al., 1982, J. Am. Chem. Soc. 104: 976; Viari, et al., 1987, Biomed. Enciron. Mass Spectrom. 14: 83; Grotjahn et al., 1982, Nuc. Acid Res. 10: 4671). Analogous sequencing methods are available for RNA oligonucleotides.

2. Competent Bacterial Expression Systems and Constructs

Nucleic acids encoding nucleocapsid antigen may be expressed in a variety of host organisms once they are operably linked in expression systems suitable for the selected host organism. Suitable expression systems typically comprise regulatory sequences operable in the host organism. These regulatory sequences are necessarily operably linked to the nucleic acid to control its expression. The expression system may optionally comprise other regulatory, replication or manipulation sequences to aid in the expression and incorporation of the nucleic acid into the expression vector, as required by the particular application being pursued.

For example, to obtain a high level expression of nucleocapsid antigen in a prokaryotic system, it is essential to construct expression vectors that contain, at a minimum; a strong promoter to direct transcription, a ribosome-binding site for translational initiation, a transcription/translation terminator, and unique restriction sites in nonessential regions of the plasmid to allow insertion of foreign nucleic acids. Other factors may also be carried on the expression vector, such as selectable and/or scorable markers, such as those described below. Suitable expression systems for use with the present invention are well-known in the art. See, e.g., Pouwels, et al. (1985 and Supplements) Cloning Vectors: A Laboratory Manual, Elsevier, N.Y.; Rodriquez, et al. (eds.) Vectors: A Survey of Molecular Cloning Vectors and Their Uses, Buttersworth, Boston, 1988; Luckow, V. A. and Summers, M. D., Bio/Technology, 6:47-55 (1988); Herskowitz, I. and Hagen, D., Ann. Rev. Genet., 14:399-445 (1980); and Yanofsky, C., J. Bacteriol., 158:1018-1024 (1984).

Exemplary bacterial host organisms suitable for use in the present invention are well-known in the art and include gram-positive and gram-negative bacteria such as Escherichia coli (cf. Sambrook et al., supra). E. coli strains are particularly preferred host organisms for expression of recombinant nucleocapsid antigen. Exemplary E. coli strains include BL21 (DE3), BL21-Gold (DE3), BL21 (DE3)-pLysS (Stratagene), MMLV-RT: JM109, DH5.alpha.f′, XL1BLUE STRATAGENE® O, San Diego, Calif.), JM105, ER 1458, NM 522, In αf′(Invitrogen, San Diego, Calif.), TOPP™. strains 1-6 (STRATAGENE®), 1200, MRE 600, Q13, and A19. Some of these strains (1200, MRE 600, Q13, and A19) are mutants that have reduced levels of RNase I (referred to as “RNase I deficient”) compared to wild type strains (Durwald et al., 1968, J. Mol. Biol. 34:331-346; Clark, 1963, Genetics 48:105-120; Gesteland, 1966, J. Mol. Biol. 16:67; Reiner, 1969, J. Bacteriol. 97:1522), while others are common laboratory strains. Some of these strains contain the lac I^{q} repressor and required use of isopropylthiogalactoside (IPTG) to induce transcription. The level of RT expression of host cells containing the RT gene was estimated by visualizing the resulting proteins on SDS-polyacrylamide gels and also, in most cases, by enzyme activity assays on crude cell lysates. Of the RNase I deficient strains, E. coli 1200 (Strain 4449, available from the E. coli Genetic Stock Center, Yale University) consistently showed high levels of enzyme expression using these assays; unless indicated otherwise, all experiments described herein were conducted using this strain.

Standard transfection methods are used to introduce expression systems for nucleocapsid antigen to host organisms. (see, e.g., Morrison, J. Bact., 132:349-351 (1977); Clark-Curtiss & Curtiss, Methods in Enzymology, 101:347-362 (Wu et al., eds, 1983); Sambrook et al., and Ausubel et al., supra.). The proteins can be recovered from the cells or from the culture medium by standard protein purification techniques described herein.

3. Selectable Marker Genes

Identifying host organisms that have successfully incorporated a nucleocapsid antigen of the present invention is preferably accomplished through inclusion of a selectable marker gene into the vector or expression system used for producing the nucleocapsid antigen coding sequence. Selectable markers allow a transformed cell, tissue or animal to be identified and isolated by selecting or screening the engineered material for traits encoded by the marker genes present on the transforming DNA. For instance, selection may be performed by growing the engineered cells on media containing inhibitory amounts of an antibiotic to which the transforming marker gene construct confers resistance. Further, transformed cells may also be identified by screening for the activities of any visible marker genes (e.g., the β-glucuronidase, green fluorescent protein, luciferase, B or Cl genes) that may be present on the recombinant nucleic acid constructs of the present invention. Such selection and screening methodologies are well known to those skilled in the art.

Physical and biochemical methods may also be used to identify a cell transformant containing the gene constructs of the present invention. These methods include but are not limited to: 1) Southern analysis or PCR amplification for detecting and determining the structure of the recombinant DNA insert; 2) Northern blot, S-1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis, western blot techniques, immunoprecipitation, or enzyme-linked immunoassays, where the gene construct products are proteins; 5) biochemical measurements of compounds produced as a consequence of the expression of the introduced gene constructs. The methods for doing all these assays are well known to those skilled in the art.

C. Protein Purification

Recombinant nucleocapsid antigen may be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells may be used for isolation of nucleocapsid antigen.

Nucleocapsid antigen expressed in bacteria may form insoluble aggregates (“inclusion bodies”). Several protocols are suitable for purification of nucleocapsid antigen from inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkman Instruments) or sonicated on ice. Alternate methods of lysing bacteria are apparent to those of skill in the art (see, e.g., Sambrook et al., supra; Ausubel et al., supra). OFP in the lysate can then be purified using standard techniques (see, e.g., Colley et al., J. Biol. Chem., 264:17619-17622 (1989); Guide to Protein Purification, in Methods in Enzymology, vol. 182 (Deutscher, ed., 1990)).

If necessary, the inclusion bodies are solubilized, and the lysed cell suspension is typically centrifuged to remove unwanted insoluble matter. Nucleocapsid antigen within inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents, which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is reversible and renaturation may occur upon removal (by dialysis, for example) or dilution of the denaturant, allowing re-formation of immunologically and/or biologically active protein. Other suitable buffers are known to those skilled in the art.

Alternatively, it is possible to purify nucleocapsid antigen from the bacteria periplasm. When nucleocapsid antigen is exported into the periplasm of the bacteria, the periplasmic fraction may be isolated by cold osmotic shock in addition to other methods known to skill in the art. E.g., isolating nucleocapsid antigen from the periplasm may involve centrifuging bacterial cells to form a pellet; resuspending the pellet in a buffer containing 20% sucrose; lysing the cells, by centrifugation followed by resuspending the pellet in ice-cold 5 mM MgSO₄ and keeping the resulting preparation on ice for approximately 10 minutes. The cell suspension is then centrifuged and the supernatant decanted and saved. The nucleocapsid antigen present in the supernatant can be separated from the host proteins by standard separation techniques well known to those of skill in the art and discussed in more detail below.

D. Standard Purification Techniques

1. Ultrafiltration

The molecular weight of nucleocapsid antigen can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut-off greater than the molecular weight of the protein of interest. The nucleocapsid antigen will pass through the membrane into the filtrate. The filtrate can then be chromatographed as described below.

2. Exchange Chromatography

Nucleocapsid antigen can also be separated from other proteins on the net surface charge, hydrophobicity, and affinity for ligands. In addition, antibodies raised against nucleocapsid antigen can be conjugated to column matrices and the antigen immunopurified. All of these methods are well known in the art. It will be apparent to one of skill that chromatographic techniques can be performed at any scale and using equipment from many different manufacturers (e.g., Pharmacia Biotech).

3. Tagging Techniques

“Affinity tags” can be fused to appropriate portions of the nucleocapsid antigen to assist in isolation and production. Typically, such “fusion proteins” are created by linking a nucleotide coding sequence for the affinity tag with in-frame with the nucleotide coding sequence for the nucleocapsid antigen. Affinity tags may also be fused to nucleocapsid antigen through cleavable linker sequences. For example a FLAG sequence, or functional equivalent, can be fused to the nucleocapsid antigen via a protease-removable sequence, allowing the FLAG sequence to be recognized by an affinity reagent, and the purified protein subjected to protease digestion to remove the extension. Many other equivalent affinity tags exist, e.g., glutathione-S-transferase (GST) having high affinity for gluthathione, and poly-histidine affinity tags possessing affinity for heavy metal column reagents. See, e.g., Hochuli, Chemische Industrie, 12:69-70 (1989); Hochuli, Genetic Engineering, Principle and Methods, 12:87-98 (1990), Plenum Press, N.Y.; and Crowe, et al. (1992) OIAexpress: The High Level Expression & Protein Purification System, QIAGEN, Inc. Chatsworth, Calif.; which are incorporated herein by reference.

4. Electrophoretic and PAGE/Blotting Purification Techniques

Nucleocapsid antigen of the present invention can be purified using native polyacrylamide gel electrophoresis. Briefly, the technique involves preparing a polyacrylamide gel slab by mixing appropriate amounts of acrylamide and bis-acrylamide in a basic buffer solution, typically Tris-HCl based, and allowing the mixture to polymerize between a pair of parallel glass plates uniformly-spaced. By modifying the amount of acrylamide added to the mixture, slabs can be optimized for separation of proteins in particular molecular weight ranges. In the case of nucleocapsid antigen, preferred acrylamide content for the gel would be between 6% and 15%, more preferably between 8% and 12%, ideally 10%. The gel is normally loaded and run in the vertical position, with protein resolution resulting by a sieving action of the gel as the proteins are driven through the gel matrix by an electrical current applied across the gel slab. (see Schagger et al., Anal. Biochem., 166:368-379 (1987)).

Band(s) containing nucleocapsid antigen are excised from the gel, and the resulting gel slices placed in a dialysis sack with the appropriate molecular weight cut-off and containing a buffer solution with a pH value preferably between 7 and 9, more preferably between 7.5 and 8.5. The sack is placed on a flat bed electrophoresis unit parallel to the direction of the current. The electrophoresis unit is filled with the same buffer solution placed in the dialysis sack. The electrophoresis unit is run for several hours, preferably overnight, at a low voltage of between 5 and 50 volts, more preferably between 15 and 30 volts (the actual voltage applied depends upon the application, particularly the composition of the buffer solution used in the apparatus).

By subjecting the gel slice containing the nucleocapsid antigen to the low voltage and current of the flat bed electrophoresis apparatus, the proteins are driven out of the gel slice and into the buffer solution of the dialysis sack. Once electrophoresis is complete, the vacant gel slices can be removed, and the nucleocapsid antigen concentrated using any one of the variety of concentration methods known in the art.

All chemicals and apparatus used in the methods noted above are described in available scientific literature and are commonly available through scientific catalogs. (see, e.g., Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series; and manufacturers' literature on use of protein purification products, e.g., Pharmacia, Piscataway, N.J., or Bio-Rad, Richmond, Calif.; and Sambrook et al., supra).

As an alternative procedure, nucleocapsid antigen resolved by the vertical gel electrophoresis method can be transferred, using Western blotting techniques commonly known in the art, to nylon or PVDF membranes, or the like. Portions of the membranes containing nucleocapsid antigen may then be isolated for identification. See Mozdzanowsky et al., Electrophoresis, 13:59-64 (1992).

III. Detecting SARS CoV Exposure Using the Present Invention.

Certain embodiments of the present invention provide methods for detection of antibodies specific for SARS CoV that are far superior in sensitivity and ease of use in comparison to currently available SARS diagnostic tests. These methods take advantage of the exceptional immunoreactivity of the nucleocapsid antigens described herein. Nucleocapsid antigens of the present invention include peptides and proteins having at least about 75%, more preferably at least about 80%, 85%, 90%, most preferably at least about 95%, 98%, 99% or 100% amino acid sequence homology with SEQ ID NO:2 or SEQ ID NO:6 or both. Moreover, nucleocapsid antigen from a variety of sources, including recombinant and synthetic, are encompassed in the present invention, with recombinant nucleocapsid antigen displaying comparable immunoreactivity with SARS-challenged patient sera to nucleocapsid antigen isolated from viral particles.

Some methods of the present invention share certain characteristics. For example, methods of the invention provide a support for immobilizing nucleocapsid antigen. In those methods, the immobilized nucleocapsid antigen is contacted with a biological sample, such as serum, from a patient suspected to have been challenged with SARS CoV. Contact of the nucleocapsid antigen with the biological sample is optionally followed by a wash step to remove loosely and non-specifically-bound material. If the patient has been challenged with SARS CoV, the patient's biological sample will contain antibodies that specifically bind nucleocapsid antigen, which has been shown to be the most immunoreactive SARS antigen (see examples section, below). Antibodies binding to the nucleocapsid antigen are then detected using a binding moiety that specifically recognizes the antibody. Exemplary binding moieties include antibodies, Fab and F(ab)₂ fragments, aptamers, and the like. In some embodiments, the binding moiety is labeled to aid in detection.

The sections immediately preceding the present discussion elaborate on the diagnostic methods of the invention and suitable labels for use in the disclosed methods.

A. Molecular Labeling

The particular label or detectable group used in assays of the present invention is not a critical aspect of the invention, as long as it does not significantly interfere with the specific binding of the nucleic acid or protein used in the assay. In those embodiments where a label is desirable, the detectable group can be any material having a detectable physical or chemical property. Such detectable labels have been well-developed and, in general, any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in commercial ELISA assays), and calorimetric labels such as colloidal gold or colored glass or plastic beads (e.g., polystyrene, polypropylene, latex, etc.).

The label may be coupled directly or indirectly according to methods well known in the art. As indicated above, a wide variety of labels may be used, with the choice of label depending on sensitivity required, ease of conjugation with the compound, stability requirements, available instrumentation, and disposal provisions.

B. Immunological Testing

The present invention provides embodiments for immunochemically-based diagnostic tests for the detection of SARS CoV exposure. These embodiments provide one of skill with the tools necessary for diagnosing SARS exposure and infection. Any nucleocapsid antigen may be used in the diagnostic tests of present invention, with the preferred antigen being SEQ ID NO:2 expressed recombinantly. Exemplary diagnostic tests are based on ELISA and Western blot formats, as described below, although one of skill in the art will recognize that the nucleocapsid antigen of the present invention may be useful in a variety of diagnostic and prognostic test procedures.

1. Sample Preparation

Any sample suspected of containing nucleocapsid antigen antibodies may be tested in accordance with the diagnostic test methods set forth herein. Preferably, the samples to be tested are bodily fluids such as whole blood, serum, plasma, cerebrospinal fluid, colostrum, lymphatic fluid, breast milk, saliva, urine, nasal wipes, tears, mucus ascites fluid, semem, fecal matter, sputum, fetal fluid and the like. Due to the sensitivity of the test described, it is both possible and preferable to strongly dilute the sample prior to testing. Dilution may proceed by addition of any fluid compatible with each of the sample, the antibodies to be tested, and the immobilized antigenic composition. Serum, when used as the sample, is preferably diluted with one or more fluids selected from the group consisting of phosphate-buffered saline, pH 7.0-7.4 (hereinafter “PBS”), PBS-containing TWEEN 20 (hereinafter, “PBS T”), PBS T with thimerosal (hereinafter, “PBS TT”), PBS TT (gelatin) (hereafter “PBS TTG”), and PBS TTG with bovine gamma globulin (hereafter “PBS TTGG”), and is preferably diluted. Preferred dilution ratios when testing for IgG antibody are about 1:50 to about 1:200. IgG tests are preferred. Preferred diluents and dilution ratios may vary according to the sample being tested.

While dilution of sample is not required, it is believed that large dilution ratios reduce the possibility that significant antigen/antibody complexes will be formed in the absence of nucleocapsid antigen-specific antibodies. The extent of dilution should be taken into account in adjusting the threshold level of antigen/antibody complex which should be considered a positive signal.

2. Nucleocapsid Antigen Preparation

Preferred antigenic mixtures include recombinant and synthetic nucleocapsid antigen, and viral extracts of varying purity where the nucleocapsid antigen fragments have an apparent molecular weight on SDS-PAGE of approximately 48,000, 44,000, or 40,000 Daltons. A mixture of antigens from an extract obtained from a SARS-infected cell is believed to include at least one nucleocapsid antigen likely to be present in almost all SARS CoV strains. Hence, a broad specificity results, enabling the antigenic mixture to be useful in serologic assays. It is preferred that the antigenic composition be enriched in nucleocapsid antigen or in at least one of the 48,000, 44,000, or 40,000 Dalton fragments. More preferably, at least 50 percent of the composition or at least 50 percent of the fragments observed by Coomassie staining after gel electrophoresis are 48,000, 44,000, or 40,000. In certain preferred embodiments the concentration reaches 85 percent or more. For some applications, it may be desirable that the antigenic composition be substantially free of contaminating antigens other than 48,000, 44,000, or 40,000 flagella or the specified molecular weight fragments.

An antigenic composition is considered to be substantially free of antigens, other than nucleocapsid antigen, whenever the antigenic composition subjected to electrophoresis on SDS-PAGE and appropriate staining exhibits single well-defined bands corresponding to known nucleocapsid antigen, and no other bands are visually apparent.

3. Fusion Proteins Including the Nucleocapsid and Spike Glycoproteins Antigens

The present invention also provides fusion proteins comprising amino acid sequence elements from both the nucleocapsid and spike glycoprotein. These fusion proteins find utility as both diagnostic and therapeutic tools for the treatment of SARS CoV infection.

Fusion proteins of the present invention include an amino acid sequence having at least about 75%, more preferably at least about 80%, 85%, 90%, most preferably at least about 95%, 98%, 99% or 100% sequence homology to SEQ ID NO:2 or SEQ ID NO:6 covalently linked to an immunogenic peptide comprising an amino acid sequence having at least about 10, more preferably at least about 12, 14 or 16, most preferably at least about 20 contiguous amino acids taken from one of the amino acid sequences SEQ ID NO:10, SEQ ID NO:14 or SEQ ID NO:18. Fusion proteins of the invention are defined functionally as having the ability to produce an immune response when introduced to a mammal intravenously. The nucleocapsid amino acid sequence may be N-terminal or C-terminal to the spike glycoproteins sequence. Synthetic linker peptides may be used to couple the SARS-specific sequences, for example a short peptide of 6 to 12 glycine residues or mixture of glycine and alanine residues may be used. Other synthetic linkers are also contemplated, and may be determined and synthesized by those of skill in the art through routine experimentation.

The fusion proteins of the present invention may be synthesized using any suitable technique known to those of skill, including solid-phase synthesis and recombinant techniques as described herein. For example, nucleocapsid and spike glycoprotein nucleotide sequences may be isolated using molecular biological techniques known in the art. The nucleotide sequences are then joined, either directly or through a nucleotide sequence encoding a linker peptide using known methods, and inserted into an expression vector, as generally described above for nucleocapsid antigen. The expression vector is then introduced into a suitable host cell, and cultured to express the fusion protein. Finally, the fusion protein is harvested and optionally purified from the cell culture according to methods described herein and well-known in the art.

Techniques for evaluating immune responses elicited by immunogenic peptides are well-known in the art. For purposes of the invention, a fusion protein is said to elicit an immune response in an animal when a challenged animal produces antibodies specifically recognizing a SARS CoV spike glycoprotein and the nucleocapsid. “Specific recognition” occurs when sera from the challenged animal recognizes the spike glycoprotein and the nucleocapsid with at least two times, more preferably three times, most preferably 5 times, ideally 10 times greater affinity than the sera recognizes a standard antigenic protein that has not been introduced to the animal. Specific recognition is tested using standard ELISA techniques employing buffers of physiologic pH and ionic strength (i.e. about pH 7.2 and 0.1N ionic strength) at room temperature.

Fusion proteins of the present invention may be utilized identically to those applications described herein for the nucleocapsid antigen. For example, the fusion protein can be used as the antigenic reagent in methods for detecting exposure to SARS-CoV using a sample from a patient, and as the active reagent in immunostimulatory preparations and vaccines.

4. General Methodology for Immunochemical Testing

For immunochemical test procedures of the invention, nucleocapsid antigen in accordance with the present invention is preferably immobilized on a solid support using conventional techniques. For instance, polystyrene plates may be incubated with nucleocapsid antigen made in accordance with the invention. Alternatively, for instance, nucleocapsid antigen isolated as protein bands on electrophoretic gel may be transferred to a nitrocellulose sheet by known methods. See Towbin et al., Proc. Nat'l. Acad. Sci., 76: 4350-54 (1979); Burnette, et al., Biochem., 112: 195-203 (1981). Numerous other techniques are known in the art for binding antigens to substantially inert substrates.

Bound antigens in accordance with the invention are preferably contacted with a highly dilute fluid that includes the sample to be tested for presence of antibody to SARS CoV. The nucleocapsid antigen and sample are preferably incubated for at least about one hour. Considerably less time is needed when incubation proceeds at or near human body temperature, about 37° C. Incubation at other temperatures, for instance 4° C., is also proper, but generally requires additional incubation time. The preferred incubation time at 37° C. is from about 10 minutes to about 90 minutes. The bound antigens should then be rinsed to remove any unbound antibodies, i.e., those that are not specific for the antigens. Preferably, rinsing proceeds with a buffer solution such as PBS T, PBS TT or Tris/TWEEN/Sodium chloride/azide. Multiple washings are preferred.

During incubation, nucleocapsid antigen-specific antibodies bind to the immobilized nucleocapsid antigen to create antigen/antibody complexes. All unbound antibodies are substantially removed during the washing procedure. Due to the high specificity of the nucleocapsid antigen of the invention, antibodies that are not specific for nucleocapsid antigen have been substantially removed at this point. Naturally, if the tested sample did not contain nucleocapsid antigen-specific antibodies, the immobilized antigens would be substantially free of human antibody and subsequent testing for antigen/antibody complexes should be negative for such complexes.

Detection of antigen/antibody complex may be achieved by a wide variety of known methods. Preferred methods include but are not limited to enzyme-linked immunosorbent assay, Western blot technique or indirect fluorescence assay. In one embodiment, a liposome based assay may be used, wherein antigen recognized by nucleocapsid antigen-specific antibody is expressed onto a liposome and binds nucleocapsid antigen-specific antibody for subsequent detection as explained in more detail below.

Typically, the nucleocapsid antigen-specific antibodies complexed with immobilized nucleocapsid antigen are detected by contact with labeled or otherwise detectable second antibodies specific for human immunoglobulin. The labeled second antibodies may be specific for any human antibody, preferably of the IgG or IgA type, most preferably, IgG. When acute sero-conversion is suspected, an IgM test may be appropriate. The second antibodies are preferably incubated with the immobilized antigens for about 15 minutes to about 2 hours, preferably 30 minutes to 60 minutes at a temperature of about 20° C. to about 37° C. Nucleocapsid antigen is then washed with a buffer solution (preferably multiple times) in order to remove all unbound labeled antibody. At this point, labeled antibody has been substantially removed except where it has bound to human immunoglobulin present on the antigens. The presence of nucleocapsid antigen-specific antibody may be indirectly measured by determining the presence or absence of the labeled second antibody. There are many known techniques for detecting the label. For instance, fluorescein-labeled antibody may be detected by scanning for emitted light at the characteristic wavelength for fluorescein. Alternatively, an enzyme label is detected by incubation with appropriate substrate and detection of a color change. This can be determined by visual inspection or can be read automatically by a spectrophotometer set at the appropriate wavelength. In Western blotting, for example, the positive signal may be detected when an enzyme is conjugated to the second antibody. Incubation with appropriate substrate enzymatically produces a color product in the immediate vicinity of the antigenic band resolved by this process. The presence of a reactive band may be detected by visual inspection. In an indirect immunofluorescence assay, fluorescein-labeled second antibodies may be detected by fluorescence-activated detectors, or by visual inspection.

A liposome-based assay may involve the presence of fluorescein, an enzyme or a substrate inside a liposome onto which surface nucleocapsid antigen is displayed. Liposomes are incubated with the body fluid sample to be tested, in appropriate dilution, and are thoroughly washed. Liposomes with human immunoglobulins on their surface forming an antigen/antibody complex may be recognized by incorporating a second antibody to a specific human Ig onto the inside walls of a polystyrene tube. Those liposomes with antibody bound to nucleocapsid antigen will be immobilized, and non-immobilized liposomes will be washed away. The liposomes can be lysed with, for instance, detergent, or complement, and the enzyme or substrate that was in the interior is now free to react with the complementary substrate (or enzyme) in the solution in the tube. The resulting color reaction could be detected by visual inspection or spectrophotometric color determination. Alternatively, fluorescein present could be detected by a fluorescence-activated detector.

The sensitivity and specificity of the antibody detection in accordance with the present invention have been determined using serum obtained from persons from defined populations. These results are graphically displayed in FIG. 2 and discussed in the examples section of this specification.

Two exemplary assay formats following the general protocol described above are the Western blot assay and the ELISA assay. Each of these assay formats is discussed in greater detail, below and in the examples section of this specification.

C. Western Blot (WB) Method

As noted above, exemplary embodiments of immunochemically-based diagnostic assays include those based on western blot methodology, as known by those of skill in the art. Although nucleocapsid antigen preparations for use in the invention are generally available as complex mixtures, such as cell lysates of SARS CoV-infected cells, the present invention preferably provides assays where the nucleocapsid antigen is recombinant, most preferably purified to homogeneity or near homogeneity using techniques known to those of skill in the art.

In a traditional Western blot, an antigenic mixture of interest is solubilized, usually with sodium dodecyl sulfate (SDS), urea, and, alternatively, with reducing agents such as 2-mercaptoethanol. Following solubilization, the material is separated, for example, on a polyacrylamide gel by electrophoresis. Antigens are then electrophoretically transferred to a solid support, such as nitrocellulose paper, where they are bound irreversibly. This procedure is described by Gordon et al., U.S. Pat. No. 4,452,901 issued Jun. 5, 1984.

The electrophoretic transfer of the proteins gives a faithful replica of the arrangement of the excised gels on a suitable solid support. The antibody assays with such transferred electrophorograms are carried out after the residual adsorption capacities of the solid support have been saturated by incubation with a non-specific protein. Immunoassays with electrophoretically transferred proteins are possible because no exchange takes place between the electrophoretically blotted specific proteins and the non-specific proteins used for blocking the residual binding sites of the support. The lack of interference of bound antigens with the non-specific proteins used for blocking the residual adsorption sites allows for prolonged incubation periods because further contact with the antisera and the indicator antibody do not generate side-reactions, such as exchange with the adsorbed non-specific proteins.

The solid support may be any material with sufficient surface porosity to allow access by detection antibodies and a suitable surface affinity to bid nucleocapsid antigen. Microporous structures are generally preferred, but materials with gel structure in the hydrated state may be used as well. Useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatin; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylates, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, magnesium carbonate, silicates of alkali and alkaline earth metals, aluminum and magnesium; and aluminum or silicon oxides or hydrates, such as clays, alumina, talc, kaolin, zeolite, silica gel, or glass (These materials may be used as fillers with the above polymeric materials); and mixtures or copolymers of the above classes, such as graft copolymers obtained by initiating polymerization of synthetic polymers on a pre-existing natural polymer.

All these materials may be used in suitable shapes, such as films, sheets, or plates, or they may be coated onto or bonded or laminated to appropriate inert carriers, such as paper, glass, plastic films, or fabrics.

The solid support is preferably in the form of sheets of thickness from about 0.01 to 0.5 mm, preferably about 0.1 mm. The pore size may vary within wide limits, and is preferably from about 0.025 to 15 microns, especially from about 0.15 to 15 microns.

The surfaces of these supports may be activated by chemical processes that cause covalent linkage of the antigens or immunoglobulins to the support. The irreversible binding of the antigen or antibody is obtained, however, in general, by adsorption on the porous material by poorly understood hydrophobic forces. A preferred support based on nitrocellulose is sold under the trade name Millipore® by the firm Millipore, Bedford, Mass., USA. Suitable supports are also described in U.S. patent application Ser. No. 7/227,272 filed Aug. 2, 1988, hereby incorporated by reference.

Once nucleocapsid antigen has been bound to the solid porous support, the support must be processed to block excess binding sites of the porous material before being usable for immunoassays. This is done by incubation of the support containing the antigenic polypeptides with non-specific proteins or with a mixture of such proteins, or with total serum from an individual that has not been challenged with SARS CoV, or any combination of these ingredients alone or together. The only limitation is that the proteins should not interfere or cross-react with any of the antibodies or nucleocapsid antigen in the immunoassays, and that they be different from proteins mounted on the support. Blocking of residual adsorption sites may also be made in steps.

For example, in a preliminary step the support containing the fixed nucleocapsid antigen may be incubated with proteinaceous material. Such proteins are advantageously diluted in buffer and incubated with the support. After this preliminary treatment there may still be binding sites present that have not been completely blocked but should be blocked before immunoassays are carried out. If there is background adsorption due to remaining binding sites, it may be prevented by carrying out the incubation with additional blocking agents. The presence of these mixtures both blocks remaining binding sites, and tends to prevent, by competition, exchange of antibodies with proteins previously bound to non-specific sites, or non-specific interaction of any kind with immunoglobulins.

To detect antibodies recognizing the SARS CoV nucleocapsid antigen, the solid support is incubated with a sample diluted in blocking solution according to the expected antibody concentration, usually from about 1:50 to 1:200, for about 2 hours at room temperature or overnight at 4. ° C., and then washed with buffer to remove excess unbound antibodies. The support is then incubated with a detectable binding moiety, for example an indicator antibody that is radioactively, fluorescently, luminescently labelled, or conjugated with an enzyme capable of producing a color reaction with an appropriate substrate, as described above. The indicator antibody is usually diluted in a mixture of the blocking solution, incubated with the support for about two hours, and washed again in buffer. Suitable samples that may contain antibodies recognizing the SARS CoV antigen include whole blood, serum, plasma, cerebrospinal fluid, colostrum, lymphatic fluid, breast milk, saliva, urine, nasal wipes, tears, mucus ascites fluid, semem, fecal matter, sputum, fetal fluid and the like.

Detection of anti-nucleocapsid antigen antibodies on the support may be made with a suitable indicator antibody, or with a component of the complement system, or with a coupled enzyme system which is sensitive to the nucleocapsid antigen-antibody reaction. Suitable indicator antibodies may be any antibody that will react specifically with human or animal immunogobulins, or class specific antibodies that react only with one desired antibody class such as IgG, IgM or IgA, or any desired combination of such specific immunoglobulins.

D. ELISA Method

Preferred immunoassays of the invention include various types of enzyme linked immunosorbent assays (ELISAs) known to the art, with a particularly preferred embodiment described in the examples section, below. The procedure for performing ELISA assays of the present invention is analogous to procedures previously described for nucleocapsid antigen bound to a solid support as, for example, in the Western blot assay described above. Briefly, nucleocapsid antigen, preferably SEQ ID NO:2, or appropriate peptides incorporating nucleocapsid antigen sequences are immobilized onto a solid support, preferably a surface exhibiting a protein affinity such as the wells of a polystyrene microtiter plate. Other exemplary solid supports suitable for the ELISA assays of the present invention are described above. By way of example, about 10 μl of test biological sample (patient sample) in about 90 μl of a suitable buffer solution (e.g., PBS with about 1% digitonin or other mild protein solubilizing agent) may be placed in each microtiter well. Control wells may include normal sera (e.g., human sera known to be free of anti-nucleocapsid antigen antibody.

As in previously described assays, bound nucleocapsid antigen may be washed to remove incompletely adsorbed material, one will desire to bind or coat a nonspecific protein such as bovine serum albumin (BSA), casein, solutions of milk powder, gelatin, PVP, superblock, or horse albumin onto the well that is known to be antigenically neutral with regard to the patient sample to be tested. As noted above, this allows for blocking of nonspecific adsorption sites on the immobilizing surface and thus reduces the background caused by nonspecific binding of antibodies from the patient sample onto the surface. Following an appropriate coating period (for example, 3 hours), the coated wells are rinsed several times (e.g., 4 or 5 times) with a suitable buffer, such as PBS. The wells of the plates may then be allowed to dry, or may instead be used while they are still wet.

The immobilizing surface is then contacted with the patient sample to be tested in a manner conducive to immune complex (antigen/antibody) formation. Such conditions preferably include diluting the antisera with diluents such as BSA, bovine gamma globulin (BGG) and phosphate buffered saline (PBS)/Tween. These added agents also tend to assist in the reduction of nonspecific background. The patient sample is allowed to incubate 15 minutes to 4 hours, at preferably about 20° C. to about 25° C., although other temperature/time combinations are suitable and may be determined by one of skill in the art through routine experimentation. Following incubation, samples are preferably washed to remove extraneous material. A preferred washing procedure includes washing with a solution such as PBS/Tween, or borate buffer.

Presence of anti-nucleocapsid antigen antibody in the patient sample then may be determined by treatment with a second antibody having specificity for the anti-nucleocapsid antigen antibody, in an analogous manner to that described above. The second antibody will preferably be an antibody having specificity in general for human IgG, IgM or IgA. The second antibody will preferably be associated with a label to aid detection, preferably an associated enzyme that will generate a color development upon incubating with an appropriate chromogenic substrate. Suitable labels for use in the present invention are known in the art, with preferred labels of the invention providing quantitative determination of the amount of anti-nucleocapsid antigen antibody present in the sample. By way of example, the amount of an enzymatic label may be quantified by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

IV. Prophylactic Medicaments for Treating SARS Infection

Nucleocapsid antigen of the present invention also finds utility as a component in prophylactic medicaments suitable for use in delaying symptomatic SARS, preferably preventing SARS CoV infection entirely. Certain prophylactic medicament embodiments of the present invention take the form of peptide-based vaccines suitable for administration to humans and promoting an immune response sufficient to inhibit or prevent nascent viral infection. Other medicaments of the invention are live vaccines. Each of these embodiments is discussed in greater detail, below.

1. Formulating Pharmaceutical Compositions

It will be appreciated that the vaccine of the invention may be useful in the fields of human medicine and veterinary medicine. Thus, the subject to be immunized may be a human or other animal, for example, farm animals including cows, sheep, pigs, horses, goats and poultry (e.g., chickens, turkeys, ducks, fowl, game birds and geese) companion animals such as dogs and cats; exotic and/or zoo animals; and laboratory animals including mice, rats, rabbits, guinea pigs, and hamsters.

For use as a medicament, nucleocapsid antigen of the invention may be used alone or conjugated to other molecules. One conjugated nucleocapsid antigen embodiment includes lipids that have been identified as agents capable of assisting the priming CTL in vivo against viral antigens. By way of example, palmitic acid residues can be attached to the alpha and epsilon amino groups of a Lys residue and then linked, e.g., via one or more linking residues such as Gly, Gly-Gly-, Ser, Ser-Ser, or the like, to nucleocapsid antigen. The lipidated antigen can then be injected directly in a micellar form, incorporated into a liposome or emulsified in an adjuvant, e.g., incomplete Freund's adjuvant. In a preferred embodiment a particularly effective immunogen comprises palmitic acid attached to alpha and epsilon amino groups of Lys, which is attached via linkage, e.g., Ser-Ser, to the amino terminus of the nucleocapsid antigen.

As another example of lipid priming of CTL responses, E. coli lipoproteins, such as tripalmitoyl-S-glycerylcysteinlyseryl-serine (P3 CSS) can be used to prime virus specific CTL when covalently attached to an appropriate peptide. See, Deres et al., Nature 342:561-564 (1989), incorporated herein by reference. Nucleocapsid antigen of the invention can be coupled to P3 CSS, for example, and the lipopeptide administered to an individual to specifically prime a CTL response to the target antigen. Further, as the induction of neutralizing antibodies can also be primed with P3 CSS conjugated to a peptide that displays an appropriate epitope, the two compositions can be combined to more effectively elicit both humoral and cell-mediated responses to infection.

Those skilled in the art of preparing pharmaceutical compositions will realize how to prepare the peptides and conjugates described above for pharmaceutical use in composition comprising accepted pharmaceutical carriers, particularly vaccines.

Nucleocapsid antigen may also be bound to a carrier protein, according to methods known in the art. See, for instance, M. F. Good, Science 235:1059-1062 (1987); and Palker, T. J., J. Immunol. 142:3612-3619 (1989). Agents that can be conjugated to nucleocapsid antigen to provoke an immune response include toxoids such as diphtheria toxoid or tetanus toxoids, which are commonly recognized by the body (of immunized persons) and eliminated by the immune system. Alternatively, a nucleotide sequence encoding nucleocapsid antigen may be incorporated into a recombinant gene and expressed as part of a vector, for instance, a recombinant virus such as vaccinia virus made by the method of Chakrabarti, S., et al., Nature 320:535-537 (1986).

Nucleocapsid antigen also may be incorporated into a larger peptide comprising additional epitopes, either other T cell epitopes or B cell epitopes. Thus, for example, nucleocapsid antigen may be used as part of a multivalent vaccine that induces cytotoxic T cell responses to multiple epitopes of SARS CoV or of SARS CoV and another virus. In addition, the multivalent vaccine peptide may include helper T cell epitopes and B cell epitopes of SARS CoV or another virus, to effect induction of an antibody response as well as a cytotoxic T cell response. For instance, one could attach a helper T cell epitope from HIV, such as those described in Cease K. B., et al., Proc. Natl. Acad. Sci. USA 84:4249-4253 (1987), to provide T cell help for the CTL response. For peptides generating antiviral cytotoxic T lymphocytes, Hart, M. K., et al., Proc Natl Acad Sci USA 88:9448-9452 (1991); and for peptides inducing an antibody response, Hart M., K., et al., J. Immunol. 145:2677-2685 (1990). Collett, N. S., V. Moennig, and M. C. Horzinek. 1989. Recent advances in pestivirus research. J. Gen. Virol. 70:253-266.

The spike antigen has been found to be of relatively poor immunogenicity. Thus, it is an objective of the present invention to provide spike fusion proteins of higher immunogenicity. In one embodiment of the present invention, the nucleocapsid antigen is combined with the spike antigen of the SARS-CoV resulting in a nucleocapsid-spike fusion protein. In this fusion protein, the nucleocapsid antigen provides an adjuvant effect for the spike antigen. Fusions between the nucleocapsid antigen and the spike antigen are made as described herein.

In another embodiment of the present invention, the spike protein (rSa, rSb or rSc), which may be glycosylated, is chemically conjugated to other immunogenic carriers such as tetanus toxoid (TT) diphtheria toxoid (DT) or other proteins. Glycosylated spike proteins may be obtained from cell cultures using eukaryotic cells that contain enzymes for glycosylation. Those cell lines are known in the art. Unglycosylated spike protein may be obtained from a bacterial culture.

The principle of using TT or DT or other proteins is the same as using the nucleocapsid, which is to provide T cell epitopes for an adjuvant effect, while the B cell epitopes reside in the spike glycoprotein itself. In the scheme described here for making anti-spike antibodies, both B cells and T cells are necessary and when activated via the respective antigen-associated epitope, they co-operate in the process. TT and DT have been used for a similar purpose in bacterial vaccines to make the capsular antigens derived from respiratory disease-causing bacteria (notably, the pneumococcus, the meningococcus and Haemophilus influenzae) more immunogenic in infants (Posfay-Barbe, KM and Wald, E R, Curr. Opin. Infect. Dis. 2004, 17(3): 177-84; Rennels et al., Pediatr. Infect Dis. J. 2004, 23(5): 429-35, the disclosures of which are incorporated by reference). This is because the immune system in infants is immature and hence unable to respond to polysaccharides including the capsular antigens, but is otherwise equipped to make antibodies to proteins. The concept of using protein conjugates for the viral antigens in SARS has not been entertained hitherto because the immunogenicity of these antigens has not been questioned or addressed. Here, we emphasize the potential advantages of coupling the viral spike protein (S) to TT or DT (or other proteins).

TT or DT are strong immunogens which can help S to become more immunogenic in all individuals. Further, TT or DT can help to make S immunogenic in infants who, because of their immature immune system, may not be able to respond to the glycosylated S protein. Currently TT and DT are used routinely as childhood vaccines in Hong Kong and most parts of the world and, as such, many individuals are already primed to these toxoids. This means that these individuals are likely to respond readily and quickly to a conjugate of these toxoids.

Most importantly, the use of TT or DT may obviate the possibility that a SARS vaccine, comprising a potent SARS viral antigen, may potentiate disease rather than protect against the disease. This is an unknown risk that becomes apparent only when a vaccine is used and the disease comes again. This is different from the immediate toxicity a vaccine has, such as the induction of fever or pain. The respiratory syncytial virus (RSV) vaccine is a notorious example of a disease-enhancing vaccine (Johnson, TR and Graham, BS, .Pediatr. Infect. Dis. J. 2004, 23 (Suppl): S46-57, the disclosures of which is incorporated by reference). Caution is warranted for SARS because it is largely an immunopathological disease resulting from an exaggerated immune response, and more specifically, one involving the T cells. By using TT or DT as the carrier, stimulating T cell responses to the viral antigens may be avoided.

Thus, in a preferred embodiment of the present invention the glycosylated spike protein rSa is chemically conjugated to tetanus toxoid (TT). In another preferred embodiment of the present invention, the glycosylated spike protein rSa is chemically conjugated to diphtheria toxoid (DT).

Thus, in a preferred embodiment of the present invention the glycosylated spike protein rSb is chemically conjugated to tetanus toxoid (TT). In another preferred embodiment of the present invention, the glycosylated spike protein rSb is chemically conjugated to diphtheria toxoid (DT).

Thus, in a preferred embodiment of the present invention the glycosylated spike protein rSc is chemically conjugated to tetanus toxoid (TT). In another preferred embodiment of the present invention, the glycosylated spike protein rSc is chemically conjugated to diphtheria toxoid (DT).

Alternatively, the glycosylated spike protein (rSa, rSb or rSc) obtained from the cell culture can be chemically conjugated to other immunogenic carriers such as CpG-oligonucleotides (CpG) (Tighe et al., Eur. J. Immunol. 2000, 30(7): 1939-47; the disclosures of which is incorporated by reference).

CpG was discovered recently as a potent adjuvant for the immune system. It was originally used as a separate compound from the antigen of interest in the vaccine concoction, but recently, it has been used in experiments where it was conjugated to an antigen (Tighe et al., Eur. J. Immunol. 2000, 30(7): 1939-47). Its action resides in its ability to stimulate the antigen-presenting cells (notably, dendritic cells) through the Toll-like (9) receptor, and mediating a Th1 response.

Thus, it is an objective of this invention to provide SARS viral antigens conjugated to CpG. In a preferred embodiment of the present invention the glycosylated spike protein rSa is chemically conjugated to CpG. In another preferred embodiment of the present invention the glycosylated spike protein rSb is chemically conjugated to CpG. In yet another preferred embodiment of the present invention the glycosylated spike protein rSc is chemically conjugated to CpG.

Again, the rationale here is to make the weak S antigen more immunogenic. Here, the S antigen provides both the B and T cell epitopes to the immune system. However, in some conjugates produced the S antigen provides only the B cell epitopes but not the T cell epitopes. This is to prevent the possibility of causing disease enhancement. In such a case, the S antigen needs to be devoid of T cell epitopes (such as being composed of the minimum sequence). CpG, in such a conjugate, does not stimulate the dendritic cells. Rather, it helps the S antigen to co-stimulate the same B cell via the B cell-receptor and the Toll-like (9) receptor, respectively. The antibodies produced are expected to be of the IgM class, and these act as the first line of defense. No T cells will be involved, there is presumably no associated inflammatory response and there will be no memory response.

For purposes of the present invention, nucleocapsid antigen suitable for vaccine formulations of the present invention is any peptide or protein comprising an amino acid sequence having at least about 75%, more preferably at least about 80%, 85%, 90%, most preferably at least about 95%, 98%, 99% or 100% amino acid sequence homology with SEQ ID NO:2 or SEQ ID NO:6.

For purposes of the present invention, the spike antigen suitable for vaccine formulations of the present invention is any peptide or protein comprising an amino acid sequence having at least about 75%, more preferably at least about 80%, 85%, 90%, most preferably at least about 95%, 98%, 99% or 100% amino acid sequence homology with SEQ ID NO:10, SEQ ID NO:14 or SEQ ID NO:18.

Once formulated into a medicament, the vaccines of the present invention will raise an immune response at least 30%, more preferably at least 40%, 50%, 60%, 70%, 80%, 90%, or more as determined by quantitative ELISA testing or quantitative CTL assay as described in the art. Optional vaccine components may be added to the vaccines of the present invention to further enhance their therapeutic effectiveness, shelf-life or other property desirable in a therapeutic composition. Optional components include adjuvants, buffers, emulsion material and the like.

2. Administering Antigenic Peptides

Nucleocapsid antigen of the invention is intended for parenteral, topical, oral, or local administration for prophylactic and/or therapeutic treatment. Preferably, nucleocapsid antigen is administered intramuscularly, or intranasally. Methods for delivering peptide compositions directly to the lungs via nasal aerosol sprays have been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045.

In JP 309347/91 (priority Nov. 25, 1991) an orally or nasally administered immunogen composition comprising an immunogen capable of immunizing mammals using an adjuvant comprising of triglycerides with C₆₋₂₆ residue of saturated or unsaturated fatty acid.

WO 94/17827 (priority Feb. 15, 1993) describes a pharmaceutical preparation for topical administration of antigens to mammals via mucosal membranes. The adjuvant/vehicle preparation is selected from (a) polyoxyethylene sorbitan monoesters, (b) polyoxyethylene castor oil, (c) caprylic/capric glycerides, and (d) gangliosides.

Nucleocapsid antigen may be optionally administered to a patient dissolved in a pharmaceutically acceptable excipient, preferably an aqueous excipient. A variety of aqueous excipients may be used, e.g., water, buffered water, 0.4% saline, 0.3% glycine, and the like, including glycoproteins for enhanced stability, such as albumin, lipoprotein, globulin, etc. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

Methods of achieving adjuvant effect for the vaccine include the use of agents such as aluminum hydroxide or phosphate (alum), commonly used as 0.05 to 0.1 percent solution in phosphate buffered saline or QS21 which stimulates cytotoxic T-cells. Formulations with different adjuvants that enhance cellular or local immunity can also be used. The relative proportion of adjuvant to antigenic peptide can be varied over a broad range so long as both are present in effective amounts. For example, aluminum hydroxide can be present in an amount of about 0.5% of the vaccine mixture (Al₂O₃ basis).

The concentration of nucleocapsid antigen in pharmaceutical preparations can vary widely, i.e., from about 0.001% to as much as 15 or 20% by weight and will be selected primarily by fluid volumes, viscosities, etc., in accordance with the particular mode of administration selected. When utilized intramuscularly as an injection solution with the active ingredient in a therapeutically effective immunopotentiating amount of about 0.001 to 0.01% by weight. If prepared in the form of a tablet, capsule or suppository, it is preferred that the active ingredient be present in an amount of about 0.1 mg per tablet, suppository or capsule. In such form, the capsule, suppository or tablet may also contain other conventional excipients and vehicles such as fillers, starch, glucose, etc. Actual methods for preparing parenterally, orally, and topically administrable compounds will be known or apparent to those skilled in the art and are described in detail in, for example, Remington's Pharmaceutical Science, 17th ed., Mack Publishing Company, Easton, Pa. (1985), which in incorporated herein by reference.

Determination of an effective amount of nucleocapsid antigen to treat individuals infected with SARS CoV may be performed using methods routine to those of skill in the art, and discussed in detail above for pharmaceutically active nucleic acids.

Compositions of the invention may be administered to an individual already suffering from an infection in an amount sufficient to cure or at least partially arrest the disease and its complications. An amount adequate to accomplish this is defined as “therapeutically effective dose.” Amounts effective for this use will depend on the severity of the infection or disease and the weight and general state of the patient being treated, but generally range from about 0.001 mg/kg to about 5000 mg/kg host body weight of peptide per day, more commonly about 0.1 mg/kg to about 1000 mg/kg host body weight of peptide per day, usually about 0.25 mg/kg to about 100 mg/kg host body per day, more usually about 0.5 mg/kg to about 20 mg/kg host body weight per day, and preferably about 0.7 mg/kg to about 10 mg/kg host body weight per day. Maintenance dosages over a prolonged period of time may be adjusted as necessary. It must be kept in mind that the materials of the present invention may be employed in serious disease states, that is, life-threatening or potentially life threatening situations. In such cases, in view of the minimization of extraneous substances and general lack of immunogenicity when a human-derived polypeptide is employed to treat human hosts, it is possible and may be felt desirable by the treating physician to administer substantial excesses of these compositions.

In prophylactic applications, compositions containing the present invention are administered to a patient susceptible to or otherwise at risk for infection treated by the methods of the present invention. Such an amount is defined to be a “prophylactically effective dose.” In this use, the precise amounts again depend on the patient's state of health and weight, but are generally in the ranges described above for therapeutic use. Prophylactic administration may be particularly desirable for hosts that have been exposed or at risk for exposure of infectious diseases, e.g., health-care workers, travelers, family members of infected individuals, immunosuppressed persons, and the like. The peptides of the present invention may also be administered for surgical prophylaxis to lessen the risk of infectious complications and enhance the host's restorative response to blood loss.

3. Formulating of Nucleic Acid Vaccines

Nucleic acids of the present invention encoding nucleocapsid antigen may also be used as active ingredients in medicinal formulations suitable for treating SARS. For example small inhibitory RNAs (siRNA), ribozyme molecules, antisense nucleocapsid antigen cDNA sequences and sequences encoding the nucleocapsid antigen may all be formulated into prophylactic medicaments suitable for inhibiting or preventing SARS CoV infection, or into therapeutics that aiding in clearance of the virus from a patient. Methods for formulating such medicaments are well-known in the art and may be achieved through routine experimentation. (See, e.g., Woodrow et al, New Generation Vaccines: The Molecular Approach, Eds., Marcel Dekker, Inc., New York, N.Y. (1989); Cryz, Vaccines and Immunotherapy, Ed., Pergamon Press, New York, N.Y. (1991); and Levine et al, Ped. Ann., 22:719-725 (1993); Tang, D. C., et al. (1992) Nature 356:152; Fynan, E. F. et al. (1993) PNAS USA 90:11478; Donnelly, J. J. et al. (1995) Nat Med 1:583; Wang, B. et al. (1993) PNAS USA 90:4156; Davis, H. L., et al. (1993) Hum Mol Genet 2:1847; Ulmer, J. B. et al. (1993) Science 259:1745; Robinson, H. L. et al. (1993) Vaccine 11:957; Eisenbraun, M. D. et al. (1993) DNA Cell Biol 12:791; Wang, B. et al. (1994) AIDS Res Hum Retroviruses 10:S35; Coney, L. et al. (1994) Vaccine 12:1545; Sedegah, M. et al. (1994) Proc Natl Acad Sci USA 91:9866; Raz, E. et al. (1994) Proc Natl Acad Sci USA 91:9519; Xiang, Z. Q. et al. (1994) Virology 199:132.

A new class of vaccines are bacterial vector vaccines is also suitable for use as delivery vehicles for the therapeutic nucleic acids of the invention (See, Curtiss, In: New Generation Vaccines: The Molecular Approach, Ed., Marcel Dekker, Inc., New York, N.Y., pages 161-188 and 269-288 (1989); and Mims et al, In: Medical Microbiology, Eds., Mosby-Year Book Europe Ltd., London (1993)). These vaccines can enter the host, orally, intranasally or parenterally. Once gaining access to the host, the bacterial vector vaccines express an engineered prokaryotic expression cassette containing the therapeutic nucleic acid operably linked to the expression elements of the cassette. (See, e.g., New Generation Vaccines: The Molecular Approach, supra; Vaccines and Immunotherapy, supra; Hilleman, Dev. Biol. Stand., 82:3-20 (1994); Formal et al, Infect. Immun. 34:746-751 (1981); Gonzalez et al, J. Infect. Dis., 169:927-931 (1994); Stevenson et al, FEMS Lett., 28:317-320 (1985); Aggarwal et al, J. Exp. Med., 172:1083-1090 (1990); Hone et al, Microbial. Path., 5:407-418 (1988); Flynn et al, Mol. Microbiol., 4:2111-2118 (1990); Walker et al, Infect. Immun., 60:4260-4268 (1992); Cardenas et al, Vacc., 11: 126-135 (1993); Curtiss et al, Dev. Biol. Stand., 82:23-33 (1994); Simonet et al, Infect. Immun., 62:863-867 (1994); Charbit et al, Vacc., 11:1221-1228 (1993); Turner et al, Infect. Immun., 61:5374-5380 (1993); Schodel et al, Infect. Immun., 62:1669-1676 (1994); Schodel et al, J. Immunol., 145:4317-4321 (1990); Stabel et al, Infect. Immun., 59:2941-2947 (1991); Brown, J. Infect. Dis., 155:86-92 (1987); Doggett et al, Infect. Immun., 61:1859-1866 (1993); Brett et al, Immunol., 80:306-312 (1993); Yang et al, J. Immunol., 145:2281-2285 (1990); Gao et al, Infect. Immun., 60:3780-3789 (1992); and Chatfield et al, Bio/Technology, 10:888-892 (1992)).

Methods for testing the efficacy of both nucleic acid and protein vaccines are well-known to those of skill in the art.

V. Kits

The present invention also contemplates packaging the diagnostic or pharmaceutical embodiments of the present invention into kits that aid in the practice of the invention. For example, one kit embodiment of the invention for detecting exposure to SARS-CoV includes a protein comprising an amino acid sequence having at least about 75%, more preferably at least about 80%, 85%, 90%, most preferably at least about 95%, 98%, 99% or 100% amino acid sequence homology with SEQ ID NO:2 and instructions for using the protein to detect anti-SARS antibodies in a sample. Another kit of the invention includes a nucleic acid having at least about 75%, more preferably at least about 80%, 85%, 90%, most preferably at least about 95%, 98%, 99% or 100% nucleotide sequence homology with SEQ ID NO:1. All such nucleic acids are termed “nucleocapsid coding sequences.” These nucleic acids and proteins may be associated with a solid support, such as a plastic or glass surface, polystyrene bead or the like. Other suitable solid supports are described above, with still others being obvious to those of skill in the art. Preferable solid surfaces are in the form of a dip-stick, more preferably, the nucleic acid or protein of the kit is protected on the dipstick by a housing.

Kits of the invention may optionally include a means for collecting a sample from a patient. The particular collecting means will be dependent upon the nature of the sample to be collected, but may take the form of a syringe, swab, tissue, cup, tube, or the like. Suitable samples for use with the kits of the invention are dependent on the contents of the particular kit. For example, kits containing ELISA-type assays in dipstick format include any sample containing anti-nucleocapsid antibody. Samples suitable for use with kits that include nucleocapsid coding sequences include any sample potentially containing SARS CoV nucleic acids, e.g., whole blood, serum, plasma, cerebrospinal fluid, colostrum, lymphatic fluid, breast milk, saliva, urine, nasal wipes, tears, mucus ascites fluid, semem, fecal matter, sputum, fetal fluid and the like

Kits may optionally include a binding moiety that is specific for an anti nucleocapsid antigen antibody, such as an indicator antibody, i.e., an antibody conjugated to a detectable label. This option is preferable in kit embodiments that are operated in an ELISA or ELISA-like format. Particularly preferred labels for indicator antibodies are enzymes recognizing substrates that are catalytically converted into chromogenic products.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

The present invention identifies the nucleocapsid protein of SARS CoV as the principle antigen recognized by the immune response raised in individuals challenged with SARS CoV. The present invention further defines both the N-terminal half (SEQ ID NO:2) and the C-terminal half (SEQ ID NO:6) of the nucleocapsid protein as parts recognized by the immune response. As shown and described herein, both protein parts find utility as both a diagnostic tool and a prophylactic medicament useful in the prevention of SARS infections.

### General Methodology

1. Cell Culture and Virus Production

Vero (monkey kidney fibroblast) cells were maintained in Dulbecco modified Eagle medium (DMEM, Gibco BRL) containing 5% fetal calf serum, penicillin G 100 U/ml, streptomycin 100 μg/ml at 37° C. and 5% CO₂ in a humidified tissue culture incubator. Coronavirus strain CUHK-W1, were prepared by infecting the Vero cells and harvesting the supernatant at 20-48 hours post-infection when a marked cytopathic effect (CPE) was observed. Virus was stored at −70° C. until use.

2. Preparation of Native Viral Antigens from Cell Culture

To prepare crude viral antigen, eight 75 cm² tissue culture flasks with subconfluent (˜90%) monolayer of Vero cells were inoculated each with 0.3 ml of the SARS coronavirus stock prepared as described above. At about 16-48 hour post-infection when cytopathic effect (CPE) was observed in 50% of the cell population, cells in suspension were pooled and pelleted by centrifugation for 10 min at 450×g. Cell pellet was resuspended in 2.7 ml of lysis buffer and incubated on ice for one hour with periodic mixing. The lysate was clarified by centrifugation at 2000×g for 15 min at 4° C. and the supernatant was collected, heated at 55° C. for 30 min and stored at −70° C. until use. Negative control antigens were prepared in parallel from three flasks of non-infected Vero cells using the same procedures.

To isolate specific viral antigens, crude viral antigen prepared above was separated by electrophoresis by SDS-10% PAGE and analyzed by Coomassie-blue staining and Western blotting. Gel slices that matched in size to the spike (S) and nucleocapsid related proteins (N1, N2, N3) as detected by Western blotting were excised and pooled into an elution chamber of an electro-eluter (Amika, Columbia, USA). Protein were eluted in elution buffer (Towbin buffer with 0.5% SDS) at 40V overnight at 4° C. and concentrated to about 0.5 ml by ultrafiltration using Ultrafree-15 Centrifugal Filter Device (10,000 Dalton molecular weight cut-off, Millipore).

3. Production of Recombinant Nucleocapsid Antigen

3a. Gene Construction

Total RNA from coronavirus-infected monkey kidney fibroblast were extracted with a RNA extraction kit (Qiagen) and was reverse-transcribed with random hexamers using a cDNA synthesis kit according to manufacturer's instruction (TaqMan, Applied Biosystems). The resulting cDNA was amplified by PCR with the appropriate primer pairs. Primers (Invitrogen) were designed to target the nucleocapsid gene (from 1 to 660 nucleotide and from 630 to 1266 nucleotide) of the coronavirus according to the published sequences for the SARS coronavirus strain CUHK-W1 (GenBank accession no. AY278554). The forward (F) primers contained a BamHI site (underlined) and the reverse (R) primers a EcoR1 site (underlined) and were listed as followed:

Gene name: rNa

SEQ ID NO:3

F-BNUpp1.1
5′-CGTGGATCCATGTCTGATAATGGACCCCAA-3′

SEQ ID NO:4

R-BNLow2.1
5′-CGATGAATTCCGAGGGCAGTTTCACCACCTCC-3′

Gene name: rNb

SEQ ID NO:5

F-BNUpp3.1
5′-CGTTGGATCCGGAGGTGGTGAAACTGCCCTC-3′

SEQ ID NO:6

R-BNLow1.1
5′-CGATGAATTCTGCCTGAGTTGAATCAGCAGA-3′

PCR conditions comprised 94° C. for 3 min; 34 cycles of 94° C. 1 min, 55° C. 1 min and 72° C. 1 min, and finally 72° C. for 15 min. PCR products were analyzed on a 1% agarose gel, purified by the QIAquick gel extraction kit (Qiagen) and restriction-digested with EcoR1 and BamHI enzyme (New England Biolab). The respective digested cDNA fragments, purified by gel extraction, were ligated into the polylinker site of the BamHI/EcoR1-digested expression vector pGEX-2T (Amersham Bioscience). This vector encodes a fusion protein [27,000 Dalton glutathione S transferase (GST)] that is linked with the N-terminal of the respective recombinant viral protein. The ligated vector was transfected into the BL21 bacteria (Amersham Bioscience) by electroporation and the transformants were screened by PCR and gene sequences were confirmed by DNA sequencing.

3b. Expression as Glutathione S Transferase Fusion Protein in Bacteria

For protein expression, 100 μl overnight cultures of the respective transfectants was inoculated into 50 ml of Luria-Bertani medium supplemented with 50 μg/ml ampicillin and incubated for about 2.5 hour at 37° C. with continuous shaking. When the optical density at 600 nm of the culture reached 0.9, isopropyl-β-D-thiogalactopyranoside (IPTG) was added to a final concentration of 1 mM to induce protein expression. The bacterial culture was further incubated at room temperature for 16 hours. To isolate recombinant protein, bacterial cells were pelleted at 3500×g for 20 min at 4° C., resuspended in 2 ml of ice-cold lysis buffer and incubated on ice for 1 hour. After sonication on ice using a Branson (Danbury, Conn.) microtip sonificator at output 2-5, duty cycle 50% for three cycles (20 sec sonication and 30 sec pause), 200 μl of 10% Triton X-100 and 20 p of 0.1M DDT was added and the mixture was further incubated on ice for 30 min. The lysate was clarified by centrifugation at 11,000×g for 10 min at 4° C. and the supernatant containing the recombinant protein was harvested. To isolate recombinant protein, affinity purification was employed. 2 ml of supernatant was mixed with 100 μl of glutathione-coupled agarose (Amersham Biosciences) at room temperature for 1 hour on a roller mixer. After washing the beads with 1 ml of lysis buffer for four times, recombinant protein was eluted three times, each with 250 μl of elution buffer (20 mM reduced-form glutathione, 25 mM Tris-HCl, 100 mM NaCl, 0.1% Triton X-100, 5 mM DDT, pH 8.0) at room temperature for 10 min on a roller mixer. Pooled fractions were concentrated to about 100 μl by ultrafiltration using a Ultrafree-15 Centrifugal Filter Device (10,000 Dalton molecular weight cut-off, Millipore) according to manufacturer's instruction. Aliquots of the protein were analyzed by SDS-PAGE, Coomassie blue staining and western blot analysis and the amount determined by BCA assays (Pierce) according to manufacturer's instruction.

4. Antibody Detection Assays

4a. Western Blot Analysis

Protein samples to be analyzed were mixed with one-third volume of 4×SDS loading buffer (0.25M Tris-HCl [pH6.8], 20% 2-mercaptoethanol, 40% glycerol, 8% SDS, 0.01% bromophenol blue), heated at 100° C. for 5 min, and loaded onto a SDS-10% polyacrylamide gel. Electrophoresis was done at 150V for 80 min at room temperature using a mini-gel electrophoresis system (Bio-Rad). Proteins on gels were transferred onto a 0.22 μm PVDF membrane (Bio-Rad) at 20V for one hour in Towbin buffer (25 mM Tris, 192 mM glycine, 20% methanol, pH 8.3) using a semi-dry electro-blotting system (Bio-Rad). Blots were treated with blocking buffer (5% dry skim milk in PBS Tris buffer solution [TBS]) at room temperature for 1 hour and incubated with primary antibody (diluted human or mouse serum in dilution buffer [5% dry skim milk, 0.1% Tween 20 in TBS] at room temperature for 1 hour or 4° C. overnight. After washing twice with washing buffer (0.1% Tween 20 in PBS), secondary antibody (horseradish peroxidase-labelled anti-human antibody [IgG specific, BD Biosciences or IgM specific, Sigma] or anti-mouse antibody [Ig-specific, BD Biosciences]) at 1:2000 dilution was added and incubation was continued for 1 hour at room temperature. Blots were subsequently washed 3 times with washing buffer, each for 15 min, on a rotating platform. The immune complexes on the blots were visualized by chemiluminescence by developing with ECL plus reagents (Amersham Biosciences) and exposing to Hyperfilm-βmax (Amersham Biosciences) for 30 seconds to 3 min.

4b. Enzyme Linked Immunosorbent Assay (ELISA)

Native viral antigens (1:200 stock) or the recombinant viral antigens (5 μg/ml) all diluted in ELISA coating buffer (pH 9.6) were dispensed to 96-well microtiter plates (Immunon 2, Dynatech) and incubated overnight at 4° C. The unknown human (or mouse) serum diluted 1:50-1:200 in buffer (1.3% bovine serum albumin, 0.25% casein and 0.05% Tween 20 in PBS) was added to the washed plate. After incubating at room temperature for 15 (or 60) min, the plate was washed twice with washing buffer (0.05% Tween 20 in PBS) and 100 μl of the developing antibody (1:2000 dilution; horseradish peroxidase-labelled goat anti-human antibody [IgG or IgM specific] or goat anti-mouse Ig [polyvalent]) was added, and incubation allowed for 15 min at room temperature. Following washing for three times, 100 μl of substrate (3,3′,5,5′-tetramethylbenzidine [TMB]) was added and the plate was incubated for 15 min at room temperature. After adding 100 μl of 0.18M H₂SO₄, the OD at 450 nm was determined within 15 min using a microtiter plate reader (Dynatech).

### Example 1

### Identification of Nucleocapsid as an Important Diagnostic Antigen by Western Blot (WB) Analysis

The antibody response of SARS patients to SARS CoV challenge was analyzed to determine which antigens of the SARS virus were targeted by the patient's immune responses. A crude mixture of the viral antigens was extracted from culture cells grown with the virus and separated on a 10% polyacrylamide gel. FIG. 1A shows the gel profile of the extracted proteins after gel electrophoresis. The proteins of gel were transferred to a polyvinylidene difluoride membrane and incubated with the patient's serum, and patient's antibodies bound to transferred proteins detected using enzyme-conjugated antibodies directed against the patients antibodies and detected using a fluorometric reagent recognized by the conjugated enzyme. The method is described in the preceding paragraph (Section 4a).

As depicted in FIG. 1B, the most reactive antigens found with SARS patients but absent in non-SARS patients are located between 40 and 48 kD molecular weight, marked “N1”, “N2” and “N3”. Less reactive antigens are found at 150 kD (“S”), 80 kD and 60 kD. The molecular weight identified with the reactive antigen N1 is consistent with the nucleocapsid protein. When the sera of 46 SARS patients, 40 non-SARS pneumonia patients and 38 healthy subjects were examined using the Western blot method, the antibody response of the SARS patients was found to be directed primarily at the nucleocapsid (78% positive), and to a lesser extent, the spike protein (40%) (See FIG. 2).

### Example 2

### ELISA Studies Using Recombinant Viral Antigens to Verify the Importance of the Nucleocapsid Protein

To test that the nucleocapsid is the major SARS viral antigen recognized by a SARS-challenged immune system, recombinant antigens of the SARS nucleocapsid (the N-terminal half and the C-terminal half), spike protein (3 subunits), and non-structural proteins (NSP12 [2 subunits], NSP9 [an internal segment], NSPl3 [whole]) were produced in Escherichia coli. FIG. 3A illustrates the relationship of these molecules in the SARS viral particle. FIG. 3B illustrates the recombinantly-produced domains of each protein. FIG. 3C is a Coomassie Blue-stained gel providing a rough indication of the molecular weights of each recombinantly-expressed protein.

The recombinant antigens were subjected to ELISA analysis using patient sera, as described above. In this analysis, the recombinant N-terminal nucleocapsid antigen (rNa) was found to react with the same patient sera as the crude viral extract, providing very good discrimination between SARS and non-SARS subjects (89% sensitivity, 94% [non-SARS pneumonia]-98% [healthy subjects] specificity) (See FIG. 2). FIG. 4A confirms a strong correlation between the ELISA analysis using crude viral extract and the analysis using rNa. These results strongly suggest that the nucleocapsid is the predominant antigen present in the crude viral extract. In contrast, there was poor correlation between the rNa ELISA analysis and the subjectively-determined IF analysis performed using the same sera (FIG. 4A).

The recombinant C-terminal nucleocapsid antigen (rNb) also discriminated very well between SARS and non-SARS subjects (See FIG. 2). However, it is slightly different from rNa and it correlates less well with the crude antigen but better with the IF test (FIG. 4B).

Using both recombinant nucleocapsid antigens together (rNa+rNb), very good discrimination between SARS and non-SARS subjects was obtained (See FIG. 2), and the results approximated those of the IF test (FIG. 4C). Varying the relative proportions of rNa and rNb yielded slightly different results in terms of assay sensitivity and specificity. Ideal combinations were found in which rNb was used at much lower amounts than rNa. Best specificity, in fact, was achieved not using rNb at all in the assay. This is because 0.38% of the community population in Hong Kong who are free of SARS, or who have not encountered SARS previously or come in contact with SARS patients, have antibodies to rNb (more specifically, at the very C-terminus) but not to rNa. The reason for this reactivity is not clear but it is obviously of immense interest to the biology of SARS. Thus, since SARS patients make antibodies to both rNa and rNb, an assay comprising both rNa and rNb would yield better sensitivity than rNa alone, and would be a preferred screening test for SARS. Positive samples in the combined test can then be confirmed using rNa and rNb individually, and other types of tests, in addition to clinical judgment.

The rNa and rNb antigens can be similarly used to detect antibodies to the SARS virus or related viruses in animals, such as civet cats and other wild animals, to see if these animals carry the virus. The assay format used here is similar to that used for humans except that the developing antibody reagent used is different, which is specific for the species in question.

Moreover, ELISA analysis using N2 and N3 antigens extracted from an acrylamide gel (See, e.g., FIG. 1A) provided results quite similar to those of the rNa ELISA analysis when screening identical patient samples (See FIG. 2), suggesting that N2 and N3 have common antigenic amino acid sequence(s) with the nucleocapsid.

When the ELISA test was performed using the recombinant spike protein antigens, rSa and rSb were not reactive with patent sera, while rSc was reactive with some (13%) of the SARS sera but also with a similar proportion of non-SARS sera (FIG. 2), indicating that the spike protein is not a good source of antigen for an immunochemically-based diagnostic assay for SARS CoV. However, some improvements in reactivity was observed in an eukaryotic-derived rSA antigen, suggesting the importance of glycans in the antigenicity (data not shown). Similarly, the recombinant non-structural proteins, NSP12 (a and b) and NSP9 were not reactive with SARS sera, while NSP 13 (a methyltransferase enzyme) reacted with SARS sera in some cases but also showed cross-reaction in same cases with control sera (data not shown).

### Example 3

### Competition Analysis Using the Recombinant Protein of SEQ ID NO:2

To confirm that the N1 antigen identified in the crude viral extract presented in FIG. 1 is the nucleocapsid protein, the recombinant protein rNa (SEQ ID NO:2) was used as inhibitor in Western blot analysis. As depicted in FIG. 5A, using sera from 2 patients (S35 and S44) and each serum containing admixed rNa, rNa significantly blocked antibody interaction not only with N1, but also with the N2 and N3 antigens. A fourth antigen, N4, was also inhibited. Inhibition was greatest with N3 and N4, followed by N2, and then, N1. In contrast, when the spike antigen, rSb, was admixed with the sera, the N1-N4 activities were not affected; rather the reactivity at the 150 kD region (“S”) was completely abolished (FIG. 5A). This shows that N1-N4 are all nucleocapsid antigens, and suggests that N2, N3 and N4 are fragments of N1 in which increasing lengths of the C-terminus are lost, progressing from N2 to N4. FIG. 5A is a Western blot analysis of 2 SARS sera (#35 and #44) against the crude viral extract in the presence of various antigens (used as inhibitor). Antigens as defined in FIGS. 2 and 3; rNSP is rNSP12b. Other notations are as in FIG. 1.

FIG. 5B is a Western blot analysis of mouse sera against the crude viral extract. Sera 1, 2 and 3 were obtained individually from 3 BALB/c mice immunized (primary dose+1 booster) with rNa. U, serum from unimmunized mice (representative of 3 mice); S, serum from mice immunized (same protocol as with rNa, and using equivalent amounts of antigen) with rSa or rSb (representative of 3 mice in each group) of the antigen. The results confirm that N1-N4 are nucleocapsid antigens.

### Example 4

### Immunization of Mice with Recombinant Protein SEQ ID NO:2

Recombinant antigens, rNa, rSa and rSb, all adjoined to a carrier protein (glutathione-S-transferase or GST, used as a tag and not as an adjuvant here), were used to immunize mice, and the sera obtained from these animals used in Western blot analysis against the crude viral extract. Each mouse was injected intraperitoneally with 0.2 mg antigen per animal, in complete Freund's adjuvant, followed by a booster dose (one-fourth the primary dose or 0.05 mg) given in incomplete Freund's adjuvant 2 weeks later, also intraperitoneally. FIG. 5B shows that all 3 mice immunized with rNa produced antibodies that reacted specifically with N1, N2, N3 and N4, but with no other antigens. FIG. 5C shows that in all 3 mice, very high levels of antibodies were produced to the rNa recombinant protein and very little antibodies to the carrier protein (GST). This suggests the nucleocapsid protein is very active. In contrast, neither of the recombinant spike proteins (rSa or rSc) given in similar amounts and manner as rNa produced any reactive antibodies in any of the animals immunized (3 animals each). Moreover, FIG. 5C shows that these animals also produced very little antibodies to rSa or rSc per se as the antibodies made are mostly directed at the carrier protein. This suggests that the spike protein is not very active. In fact, the nucleocapsid antigen (rNa) could be used at a 1/10^{th} or even at 1/100^{th} concentration (0.02 mg per animal, primary dose) of that of the spike antigens (rSa or rSc) and still produced high levels of anti-nucleocapsid antibodies in the animals (data not shown). This suggests the possibility of incorporating the spike protein into the nucleocapsid protein as a vaccine, using the latter for adjuvant effect. This multi-antigenic vaccine is preferably produced in eukaryotic systems rather than in bacteria so that the spike protein is appropriately glycosylated.

1. A method of detecting exposure to SARS-CoV in a biological sample from a patient, the method comprising the steps of:
(a) contacting the biological sample to a protein comprising an amino acid sequence having at least about 75% sequence homology to SEQ ID NO:2 or SEQ ID NO:6 or both; and
(b) detecting in the biological sample an antibody binding to the contacted protein.

2. The method according to claim 1, wherein the protein comprises the amino acid sequence of SEQ ID NO:2.

3. The method according to claim 1, wherein the protein is a combination of SEQ ID NO:2 and SEQ ID NO:6 proteins used in different proportions to each other.

4. The method according to claim 3, wherein the protein is immobilized to a solid support.

5. The method according to claim 4, wherein the solid support is a plastic or a glass.

6. The method according to claim 4, wherein the solid support is selected from the group consisting of microsphere, microplate and membrane.

7. The method according to claim 1, wherein the biological sample is selected from the group consisting of whole blood, serum, plasma, cerebrospinal fluid, colostrum, lymphatic fluid, breast milk, saliva, urine, nasal wipes, tears, mucus ascites fluid, semem, fecal matter, sputum, and fetal fluid.

8. The method according to claim 1, wherein the protein comprises amino acid sequence SEQ ID NO:2 or SEQ ID NO:6 or both.

9. The method according to claim 1, wherein the protein is a recombinant protein.

10. The method according to claim 8, wherein the recombinant protein is produced in bacteria.

11. The method according to claim 1, wherein the protein comprises a segment of the amino acid sequence of SEQ ID NO:2 or SEQ ID NO:6 or both.

12. The method according to claim 1, wherein step (b) comprises:
(i) contacting the antibody bound to the protein with a labeled molecule that specifically recognizes the antibody bound to the protein; and
(ii) detecting the labeled molecule.

13. The method according to claim 12, wherein the labeled molecule comprises a label selected from the group consisting of radioactive isotopes, fluorophores, chromophores, phosphors and enzymes.

14. The method according to claim 13, wherein the label is an enzyme and the detecting step further comprises contacting the label with a molecule that is catalytically converted by the enzyme into a detectable product.

15. The method according to claim 3, wherein a detecting reagent is selected from the group consisting of colored microspheres, radioactive isotopes, fluorophores, chromophores, phosphors and enzymes.

16. The method according to claim 1, wherein the protein is a component in a molecular mixture and the method further comprises the steps of:
(c) separating the protein from other components of the molecular mixture; and
(d) transferring the protein to a solid support.

17. The method according to claim 16, wherein step (c) comprises electrophoresis of the molecular mixture through a porous support.

18. The method of claim 16, wherein the porous support is selected from the group consisting of agarose, cellulose, porous silica and polyacrylamide.

19. The method according to claim 16, wherein the solid support is selected from the group consisting of polyvinyl diflouride, nylon, cellulose and derivatives thereof.

20. A vaccine comprising:
(a) a protein comprising an amino acid sequence having at least about 75% sequence homology to SEQ ID NO:2 or SEQ ID NO:6 or both; and
(b) a pharmaceutically acceptable excipient.

21. The vaccine of claim 20, wherein the protein comprises a segment of the amino acid sequence of SEQ ID NO:2 or SEQ ID NO:6 or both.

22. The vaccine of claim 20, further comprising an adjuvant.

23. The vaccine of claim 20, wherein the protein is a fusion protein.

24. The vaccine of claim 23, wherein the protein is fused to a protein comprising an amino acid sequence having at least about 75% sequence homology to an amino acid sequence selected from the group consisting of SEQ ID NO:10, SEQ ID NO:14 and SEQ ID NO:18.

25. The vaccine of claim 23, wherein the fusion protein is produced in a eukaryotic system.

26. The vaccine of claim 23, wherein the fusion protein further comprises an immunogenic peptide comprising an amino acid sequence having at least about 10 contiguous amino acids selected from the group consisting of SEQ ID NO:10, SEQ ID NO:14 and SEQ ID NO:18.

27. The vaccine of claim 20, further comprising an antibiotic or an antiviral drug.

28. A live vaccine comprising a cell including a nucleic acid comprising a coding sequence for a first protein having at least about 75% sequence homology to SEQ ID NO:2 or SEQ ID NO:6 or both, or a fragment thereof, wherein the coding sequence is operably linked to an expression system suitable for expressing the first protein in the cell.

29. The live vaccine according to claim 28, wherein the nucleic acid further comprises a coding sequence for a second protein situated in-frame with the coding sequence of the first protein.

30. The live vaccine according to claim 29, wherein the second protein is an adjuvant.

31. The live vaccine according to claim 29, wherein the second protein is a cell surface anchor.

32. The live vaccine according to claim 28, wherein the first protein is secreted.

33. A kit for detecting exposure to SARS-CoV comprising:
(a) a protein comprising an amino acid sequence having at least about 75% sequence homology to SEQ ID NO:2 or SEQ ID NO:6 or both; and
(b) instructions for using the protein to detect an anti-SARS antibody in a biological sample.

34. The kit according to claim 33, further comprising a solid support.

35. The kit according to claim 33, further comprising one or more implements for collecting the sample selected from the group consisting of whole blood, serum, plasma, cerebrospinal fluid, colostrum, lymphatic fluid, breast milk, saliva, urine, nasal wipes, tears, mucus ascites fluid, semem, fecal matter, sputum, and fetal fluid.

36. The kit according to claim 30, wherein the amino acid sequence is SEQ ID NO:2 or SEQ ID NO:6 or both.

37. The kit according to claim 33, wherein the protein is recombinant.

38. The kit according to claim 33, further comprising a binding moiety specifically recognizing an anti-SARS antibody bound to the protein.

39. The kit according to claim 38, wherein the binding moiety is an antibody.

40. The kit according to claim 38, wherein the binding moiety is labeled.

41. The kit according to claim 38, wherein the binding moiety comprises a label selected from the group consisting of radioactive isotopes, fluorophores, chromophores, phosphors and enzymes.

42. The kit according to claim 41, wherein the label is an enzyme and the kit further comprises a molecule that is catalytically converted by the enzyme into a detectable product.

43. A diagnostic device for testing exposure to SARS CoV, the device comprising a solid support having bound thereto a protein comprising an amino acid sequence having at least about 75% sequence homology to SEQ ID NO:2 or SEQ ID NO:6 or both.

44. The diagnostic device according to claim 43, for detection of exposure to SARS-CoV in an animal.

45. The diagnostic device according to claim 43, wherein the solid support is formed as a dipstick.

46. The diagnostic device according to claim 43, wherein the solid support is enclosed within a housing.

47. A diagnostic kit comprising:
(a) a device having a solid support bound thereto a protein comprising an amino acid sequence having at least about 75% sequence homology to SEQ ID NO:2 or SEQ ID NO:6 or both; and
(b) instructions for using the device.

48. The diagnostic kit according claim 47, further comprising an antibody specifically recognizing the amino acid sequence.

49. A method of detecting exposure to SARS-CoV in a biological sample from a patient, the method comprising the steps of:
(a) contacting the biological sample to a fusion protein comprising: an amino acid sequence having at least about 75% sequence homology to SEQ ID NO:2 or SEQ ID NO:6 covalently linked to a peptide comprising an amino acid sequence having at least about 10 contiguous amino acids selected from the group consisting of SEQ ID NO:10, SEQ ID NO:14 and SEQ ID NO:18; and
(b) detecting an antibody in the biological sample binding to the contacted fusion protein.

50. An immunostimulatory preparation comprising:
(a) a fusion protein comprising an amino acid sequence having at least about 75% sequence homology to SEQ ID NO:2 or SEQ ID NO:6 covalently linked to an immunogenic peptide comprising an amino acid sequence having at least about 10 contiguous amino acids selected from the group consisting of SEQ ID NO:10, SEQ ID NO:14 and SEQ ID NO:18; and,
(b) a pharmaceutically acceptable excipient.

51. The immunostimulatory preparation according to claim 50, wherein the fusion protein further comprises tetanus toxoid, diphtheria toxoid or CpG-oligonucleotides.

52. The immunostimulatory preparation according to claim 51, wherein the tetanus toxoid, diphtheria toxoid or CpG-oligonucleotides is chemically conjugated to the immunogenic peptide.


SEQUENCE LISTING (21 sequences)

SEQ ID NO: 1 - DNA, 660 bp, severe acute respiratory syndrome (SARS) virus
  N-terminal nucleocapsid coding sequence (rNa)
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctc

SEQ ID NO: 2 - protein, 220 aa, severe acute respiratory syndrome (SARS) virus
  N-terminal nucleocapsid sequence (rNa)
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETAL

SEQ ID NO: 3 - DNA, 30 bp, Artificial Sequence
  Description of Artificial SequencePCR amplification 5′ forward (F) primer F-BNUpp1.1 for rNa
cgtggatccatgtctgataatggaccccaa

SEQ ID NO: 4 - DNA, 32 bp, Artificial Sequence
  Description of Artificial SequencePCR amplification 3′ reverse (R) primer R-BNLow2.1 for rNa
cgatgaattccgagggcagtttcaccacctcc

SEQ ID NO: 5 - DNA, 627 bp, severe acute respiratory syndrome (SARS) virus
  N-terminal nucleocapsid sequence (rNb)
ggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggca

SEQ ID NO: 6 - protein, 209 aa, severe acute respiratory syndrome (SARS) virus
  N-terminal nucleocapsid sequence (rNb)
GGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 7 - DNA, 30 bp, Artificial Sequence
  Description of Artificial SequencePCR amplification 5′ forward (F) primer F-BNUpp3.1 for rNb
cgtggatccggaggtggtgaaactgccctc

SEQ ID NO: 8 - DNA, 31 bp, Artificial Sequence
  Description of Artificial SequencePCR amplification 3′ reverse (R) primer R-BNLow1.1 for rNb
cgatgaattctgcctgagttgaatcagcaga

SEQ ID NO: 9 - DNA, 957 bp, severe acute respiratory syndrome (SARS) virus
  N-terminal spike antigen sequence (rSa)
aattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataat

SEQ ID NO: 10 - protein, 319 aa, severe acute respiratory syndrome (SARS) virus
  N-terminal spike antigen sequence (rSa)
NSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYN

SEQ ID NO: 11 - DNA, 30 bp, Artificial Sequence
  Description of Artificial Sequence5′ primer for rSa
cgtggatccaattctactaatgttgttata

SEQ ID NO: 12 - DNA, 32 bp, Artificial Sequence
  Description of Artificial Sequence3′ primer for rSa
cgatgaattccattataattaccagttgaagt

SEQ ID NO: 13 - DNA, 1365 bp, severe acute respiratory syndrome (SARS) virus
  Mid-spike antigen sequence (rSb)
acttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggca

SEQ ID NO: 14 - protein, 455 aa, severe acute respiratory syndrome (SARS) virus
  Mid-spike antigen sequence (rSb)
TSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMA

SEQ ID NO: 15 - DNA, 30 bp, Artificial Sequence
  Description of Artificial Sequence5′ primer for rSb
cgtggatccacttcaactggtaattataat

SEQ ID NO: 16 - DNA, 32 bp, Artificial Sequence
  Description of Artificial Sequence3′ primer for rSb
cgatgaattcctgccatttgcatagcaaaagg

SEQ ID NO: 17 - DNA, 948 bp, severe acute respiratory syndrome (SARS) virus
  C-terminal spike antigen sequence (rSc)
ccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatgg

SEQ ID NO: 18 - protein, 316 aa, severe acute respiratory syndrome (SARS) virus
  C-terminal spike antigen sequence (rSc)
PFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKW

SEQ ID NO: 19 - DNA, 30 bp, Artificial Sequence
  Description of Artificial Sequence5′ primer for rSc
cgtggatccccttttgctatgcaaatggca

SEQ ID NO: 20 - DNA, 32 bp, Artificial Sequence
  Description of Artificial Sequence3′ primer for rSc
cgatgaattccccatttaatatattgctcata

SEQ ID NO: 21 - protein, 12 aa, Artificial Sequence
  Description of Artificial Sequencesynthetic linker peptide
GGGGGGGGGGGG